(19) United States
(12) Patent Application Publication
(10) Pub. No.: US 2025/0172313 A1
(43) Pub. Date: May 29, 2025

(54) APPARATUS AND SYSTEM FOR AIR PURIFICATION INDOOR AIR QUALITY MONITORING, AND METHODS FOR IMPLEMENTING THE SAME

(71) Applicant: Energy Cloud Inc., San Diego, CA (US)

(72) Inventor: John CARRIERI, San Diego, CA (US)

(21) Appl. No.: 18/959,496

(22) Filed: Nov. 25, 2024

Related U.S. Application Data
(60) Provisional application No. 63/602,595, filed on Nov. 25, 2023.

(51) Int. Cl. F24F 11/88; F24F 8/24

(52) U.S. Cl. CPC F24F 11/88; F24F 8/24

(57) ABSTRACT

In one aspect, disclosed herein are embodiments of devices, system, and methods for employing the same for the purpose of monitoring, measuring, testing, and reporting on the effectiveness of Heating, Ventilation, & Air Conditioning (HVAC) and/or air purification (AP) systems for deactivating airborne biologicals, removing dangerous volatile organic compounds (VOCs), and filtering other harmful particulate matter from air. The disclosed air purification devices were employed in a specially designed test system referred herein as the Airborne Biological Assessment Threat Elimination (ABATE). Each air purification unit tested was shown to be able to clarify air traversing through the ducting of the ABATE test system of at least 99.99% of particles and viruses.

### CROSS-REFERENCE TO RELATED APPLICATIONS

This Application claims priority to and the benefit of U.S. Provisional Patent Application No. 63/602,595, titled “CLOUD BASED HVAC MANAGEMENT APPARATUS AND SYSTEM FOR AIR PURIFICATION, INDOOR AIR QUALITY MONITORING, AND METHODS FOR IMPLEMENTING THE SAME,” and filed on Nov. 25, 2023, the disclosure of which is incorporated herein by reference in its entirety for all purposes.

### FIELD OF THE INVENTION

The present disclosure is directed to the fields of air-purification systems (APS) and Heating, Ventilation, & Air Conditioning (HVAC) systems as well as an Airborne Biological Assessment Threat Elimination (ABATE) system that was designed for testing the same. Specifically, provided herein is a uniquely designed combined air-purification and intelligent HVAC system that employs photocatalytic oxidation as a mechanism for purifying and/or conditioning air. Also provided are systems and methodologies for testing the same. Specifically, presented herein is a building level air purification system that was tested in accordance with the herein described ABATE technology, whereby it was found that the tested air purification systems were found to be 99.99% effective for decomposing and deactivating airborne biologicals, such as at large infectious doses, e.g., of 3000×, at normal air flow velocities, up to 2,000 Cubic Feet per Minute per unit, thereby, preventing indoor airborne transmission.

### BACKGROUND

As is widely known, in 2019 a novel coronavirus SARS-CoV2 (severe acute respiratory syndrome coronavirus 2) was found to infect a cluster of patients in Wuhan China. This new virus SARS-CoV2 and its associated disease went on to create a worldwide pandemic named COVID-19. It is accepted that once this novel Corona Virus escaped Wuhan, it rapidly spread across the world to devastating effect. Upon performing research on this novel virus, it was determined to be different than both MERV-CoV2 and SARS-CoV.

Several studies demonstrated that the virus could be transmitted from respiratory emissions such as droplets and aerosolized breathing. Additional studies indicated that the virus could spread beyond 6 feet around an infected individual, such as through air currents and through HVAC systems. For instance, certain studies and subsequent reports sampled air and surfaces in Covid-19 patient rooms, as well as hallways outside of those rooms, and demonstrated evidence of the virus on window ledges, under patient beds, in the hallways outside of patient rooms, and up on the room's HVAC vents.

However, testing with a dangerous infectious virus or similar bio-threat is normally done in a BSL (bio safety level) lab. The labs are rated at a level 1-4 with the level indicating the protective measures to protect the lab workers, the environment and the public. “BSL-3 laboratories are used to study infectious agents or toxins that may be transmitted through the air and cause potentially lethal infection through inhalation exposure. Researchers perform all experiments in biosafety cabinets that use carefully controlled air flow or sealed enclosures to prevent infection. BSL-4 laboratories are used to study infectious agents or toxins that pose a high risk of aerosol-transmitted laboratory infections and life-threatening disease for which no vaccine or therapy is available. The laboratories incorporate all BSL 3 features and occupy safe, isolated zones within a larger building or may be housed in a separate, dedicated building.”

In the case of studying Covid-19 a level 3 BSL is required. The issue that arose, however, was that there were no suitable means of testing SARS-CoV-2/Covid-19 in realistic airflows found in HVAC systems because in both a BSL Level 3 or BSL Level 4 lab, biosafety rules normally require all work to be done in a bio-containment cabinet. These biosafety cabinets are typically 4 ft (1,200 mm) to 6 ft (1,800 mm) long with an internal workspace height of 18-24 inches (457 mm-607 mm). This small, enclosed space limited testing for airflow to small models that are not representative of actual building HVAC airflows and therefore cannot produce realistic testing. Furthermore, almost all biosafety protocols limit or discourage aerosolization. Primary hazards to personnel working with these agents relate to autoinoculation, ingestion, and exposure to infectious aerosols. At BSL-3, more emphasis is placed on primary and secondary barriers to protect personnel in contiguous areas, the community, and the environment from exposure to potentially infectious aerosols.

Hence, a problem was identified that there is no system that can test an infectious agent, such as SARS-CoV-2, by aerosolizing it and studying it with realistic building ventilation conditions.

The present devices, systems, and their methods of use as disclosed herein address and solve these problems.

### SUMMARY

Accordingly, in one aspect, the present disclosure is directed to uniquely designed indoor air-purification modules that are configured for purifying air of airborne biologicals, such as viruses, with a 99.99% deactivation effectiveness. Consequently, in another aspect, the present disclosure is directed to an Airborne Biological Assessment Threat Elimination (ABATE) system and method for testing air purification modules so as to determine their effectiveness for deactivating harmful biologicals as well a clarifying Volatile Organic Compounds (VOCs), and other harmful particulates, from an airflow. Consequently, presented herein is an ABATE test system, as well as a method for implementing the same so as to test the effectiveness of air purification modules for deactivating harmful airborne biologicals.

Specifically, the present inventor performed testing to determine whether Covid-19, and its variants, could survive in HVAC systems, and as a result of determining that it could, developed the present ABATE devices, their configurations, and the attendant systems, which are to be deployed in methods for deactivating such airborne biologicals, e.g., viruses. Particularly, the present inventor tested the in-duct air purification systems presented herein for the purpose of deactivating Covid-19 present in the air traversing in an HVAC system. More specifically, three different versions of the disclosed air purification system, including a large industrial use, in-room portable, as well as a small automotive/aviation version, were tested against several different variants of SARS-CoV2, including: Beta, BA.2, Omicron and Delta variants, and well as RSV and the Flu.

Through these studies it was found that the present devices, systems, and their methods of use were effective for reducing, e.g., deactivating, all of the above referenced viruses tested as well as bacteria, microbes, pollen, dust, dander, allergens, spores, fungi, mold, odors, smoke, and toxic fumes, without producing any harmful ozone or biproducts.

Accordingly, in one aspect, disclosed herein are embodiments of devices, system, and methods for employing the same for the purpose of monitoring, measuring, testing, and reporting on the ability that one or more of the disclosed intelligent Heating, Ventilation, & Air Conditioning (HVAC) and/or air purification (AP) systems will be effective for deactivating airborne biologicals, removing dangerous volatile organic compounds (VOCs), and filtering other harmful particulate matter from the air. For these purposes, the air purification devices disclosed herein were employed in a specially designed test system referred to herein as the Airborne Biological Assessment Threat Elimination (ABATE). Each of the air purification units that included a photocatalytic oxidization chamber that were tested were shown to be able to clarify air traversing through the ducting of the ABATE test system of at least 99.99% of particles and viruses.

Therefore, in various embodiments, provided herein is an airborne biological assessment threat elimination (ABATE) system for collecting and measuring airborne biologicals. For instance, in various embodiments, the system may include one or more of an intelligent heating, ventilation, and air conditioning (HVAC) unit, a nebulization unit, a compressor, a modular ducting subsystem, an air purification module, as well as a plurality of sampling tubes, such as positioned on either side of the air purification module. Specifically, in one embodiment the ABATE test system includes an HVAC unit for efficiently generating an airflow within a compartmentalized environment in a manner.

The HVAC unit may be combined with an air purification module that is communicably coupled to the HVAC sub-system by a number of duct elements. Hence, the air purification module may be positioned within a set of ducts, e.g., a module of ducting, where the air purification module is placed downstream of the HVAC subsystem. In such an instance, the air purification system may be configured for clarifying the air being circulated by the HVAC system within the ducting of dangerous VOCs and other harmful particulate matter, as well as for deactivating any airborne biologicals, such as viruses, that may be present within the airflow. For these purposes the air purification module may employ one or photocatalytic oxidization (PCO) chambers, through which the air is passed during a purification process.

In another aspect, provided herein is a system for testing the ability of the HVAC and/or air purification system to effectively deactivate any viruses being present within an air flow. As indicated above, this test system disclosed herein is referenced as the airborne biological assessment threat elimination (ABATE) system. This system may include one or more of a nebulization unit, a compressor, a modular ducting system, and one or more sample probes for collecting particulate matter for testing.

Accordingly, in one embodiment, the ABATE test system may include a nebulization subsystem. For instance, the nebulization subsystem may be configured for generating an airborne biological. For such a purpose the nebulization subsystem may include a nebulization unit having a plurality of chambers, a fluid pump, as well as a plurality of translation conduits. Particularly, one of the chambers may contain a biological load, while another of the chambers may include a carrier fluid, such as a fluid of mucus or saliva, for fluidizing the biological load. A third, nebulization chamber, may also be included for receiving the biological, the carrier fluid, and air under pressure so as to nebulize and/or aerosolize the biological within the carrier fluid. A number of conduits as well as a fluid pump may be included such as for moving the various components from one pf the chambers to another.

For example, in one embodiment the nebulization chamber may be coupled to the biological load chamber by a first translation conduit, but may further be coupled to the carrier fluid by another (or the same) translation conduit. Further, the nebulization chamber may be coupled to a compressed air chamber by a third translation conduit, whereby as the biological and the carrier solution are conveyed to the nebulization chamber I addition to the pressurized air, the fluidic biological load mixture becomes nebulized and/or aerosolized. Consequently, the nebulization chamber may be configured for nebulizing the liquid biological load so as to produce a nebulized biological load. For these purposes, as indicated, a fluid pump and/or a compressor unit may be coupled to the nebulization unit such as via a suitably configured translation conduit.

Further, as indicated, a modular ducting subsystem may be provided, such as where the modular ducting subsystem is fluidically coupled to the nebulization unit and configured for receiving the aerosolized biological within the ducting system. In such instances, the modular ducting subsystem may include a first and a second ducting module. Specifically, each ducting module may contain at least two segments of one or more duct elements. In various embodiments, at least one of the duct elements of each ducting may be configured as a sample collection duct, such as for collecting one or more samples of the airborne biological.

As indicated, in various embodiments, the ducting system may be modular and therefore may be composed of interlocking duct elements that can be clipped or snapped or otherwise coupled together and moved about so as to produce a ducting system of a desired shape and size. In this regard, each duct element may be roughly square or rectangular and may be composed of a pair of opposed walls defining a cavity therebetween. In particular embodiments, a plurality of ducting modules may be employed, whereby each module forms a corresponding segment of duct modules. In such instances, the two segments of duct modules, e.g., each individual duct element therein, may have respective coupling mechanisms that are adapted for removably joining the respective duct elements of the two segments together in airtight sealings.

Furthermore, in various embodiments, each segment of duct modules may include one or more duct elements that are configured for receiving one or more of an unprocessed or processed sample, e.g., a sample of the aerosolized, airborne biological. More particularly, as indicated, at least one of the sample collection duct elements of at least one of the ducting modules is configured for receiving the aerosolized biological load from the nebulization unit, such as via another, e.g., dedicated, translation conduit. This ducting element may further be configured for directing a first portion of the aerosolized biological load through the first ducting module and into the sample collection duct element of the second ducting module. This conveyance may be in a manner whereby neither air nor one or more portions of the aerosolized biological load can escape through any of the airtight sealings.

In various embodiments, a first sampling probe may be included, such as by being coupled to the sample collection duct element of the first ducting module. In certain embodiments, the first sampling probe may have a first elongated reception tube that includes a first portion and a second portion. In various iterations, the first portion of the sample tube may extend inwards from a first of the opposed walls of the sample collection duct element of the first ducting module, and into the cavity defined thereby.

Likewise, a second portion of the sample tube may extend at least partially through and outside of the first wall. In particular embodiments, the first portion may be coupled to a shrouded collection member that is configured for capturing air as it circulates past the sample probe, and directing that air into the second portion of the reception tube, but in a manner that as the air is conveyed within the shrouded member, the airflow is drastically slowed. Further, the second portion of the sample probe tube may be coupled to a particulate measurement member such that as the captured air is passed through the measurement device the number, size, and other characteristics of particulate matter present within the airflow are characterized.

In various instances, the circulated air is passed from the first sample collection duct into the air purification unit, whereby that air may be purified. Specifically, the air purification module may be positioned between the first ducting module and the second ducting module, e.g., between the first and second sample collection ducts thereof. In such instances, the air purification module may be configured for receiving an uncaptured portion of the aerosolized biological load, for purifying the uncaptured portion of the aerosolized biological load so as to produce purified aerosolized biological load, and for translating the purified aerosolized biological load into the sample collection duct element of the second ducting module.

Consequently, a second sampling probe may be coupled to the sample collection duct element of the second ducting module such as for collecting and quantifying any particulate matter still being retained within the airflow post purification. The second sampling probe may be configured in a manner similar to the first sample probe in that it may include a similar elongated reception tube that likewise has a first and a second portion. Additionally, the first portion may extend inwards from one of the opposed walls of this second sample collection duct element and into the cavity. And like the first sampling probe, the first portion of this second sample probe may also be coupled to a shrouded collection member. In like manner, the shrouded capture member may be configured for capturing a portion of the purified air and for translating that captured air to a particulate measuring device coupled to a second portion of the probe that extends exteriorly outward from the sample collection duct element.

Accordingly, in various embodiments, provided herein is a method for using the ABATE test system, such as in a manner as described herein above for collecting airborne particulate matter both pre- and post-air purification and filtration, and comparing the two samples to determine one or more characteristics of the effects of air purification on various airborne biologicals.

The details of one or more embodiments are set forth in the accompanying drawings and the description below. Other features and advantages will be apparent from the description and drawings, and from the claims.

### BRIEF DESCRIPTION OF THE DRAWINGS

These and other aspects will now be described in detail with reference to the following drawings.

FIG. 1 provides an illustration of an elongated, stacked configuration of the ABATE testing system in accordance with the teachings of the disclosure;

FIG. 2 provides another illustration of a shortened, double-stacked configuration of the ABATE testing system in accordance with the teachings of the disclosure;

FIG. 3A provides an illustration of an embodiment of a curved top duct element for use in the ABATE testing system of FIG. 2;

FIG. 3B provides an illustration of corresponding embodiment of a curved bottom duct element for use in the ABATE testing system of FIG. 2;

FIG. 4A provides an illustration of a set of exemplary sample collection probes for use within the ducting sub-system of the ABATE system in accordance with the teachings of the disclosure;

FIG. 4B provides an illustration of an exemplary duct element including a particulate sample collection probe of FIG. 4A, as it would be deployed for use in the ABATE system;

FIG. 4C provides an illustration of another exemplary sample collection probe of FIG. 4A, this time configured as a virus collection probe;

FIG. 5 provides an illustration of an exemplary process for performing an ABATE test procedure;

FIG. 6A provides an illustration of an exemplary viral loading apparatus of a nebulization system of the disclosure;

FIG. 6B provides an illustration of a bottom, nebulization portion of the exemplary viral loading apparatus of the nebulization system of FIG. 6A;

FIG. 6C provides an illustration of an exemplary nebulizer head unit for use in accordance with the teachings of the disclosure;

FIG. 6D provides an illustration of an exemplary control unit for controlling the nebulization system of FIG. 6A;

FIG. 7A provides an illustration of another embodiment of a nebulization system of the disclosure;

FIG. 7B provides an illustration of a pair of compressor units for use in performing a nebulization operation;

FIG. 8 provides a further illustration of a shortened, double-stacked configuration of the ABATE testing system of FIG. 2, highlighting where various system measurements ay be performed;

FIG. 9A provides another illustration of an elongated, stacked configuration of the ABATE testing system of FIG. 1 in an open, single-pass configuration;

FIG. 9B provides an illustration of a ducting element of FIG. 9A, in the closed, single-pass configuration;

FIG. 9C provides a further illustration of a ducting element of FIG. 9A, but this time with transition duct being coupled to the duct exhaust;

FIG. 10A provides an illustration of an exemplar nebulization duct element of FIG. 1;

FIG. 10B provides an illustration of the exemplary collection duct of FIG. 3B, having a collection door positioned therein;

FIG. 11A provides a further illustration of an elongated, stacked configuration of the ABATE testing system of FIG. 1 where an air conditioning unit has been substituted for one or more other duct elements;

FIG. 11B provides an additional illustration of the ABATE testing system of FIG. 11A where an industrial fan unit has been substituted for the air conditioning unit of FIG. 11A;

FIG. 12 provides an additional illustration of the ABATE testing system of FIG. 1 where a turning vane has been positioned within the cavity of a corner duct element;

FIG. 13A provides a table setting forth a set of ABATE control test results of a first test that was performed in accordance with the teachings of the disclosure;

FIG. 13B provides a graph pursuant to the test results of FIG. 13A;

FIG. 14A provides a table setting forth a set of ABATE air purification test results of a second set of tests that were performed in accordance with the teachings of the disclosure;

FIG. 14B provides a graph pursuant to the tests results of FIG. 14A;

FIG. 15A provides a table setting forth a set of ABATE air purification tests results of a third set of tests that were performed in accordance with the teachings of the disclosure;

FIG. 15B provides a graph pursuant to the tests results of FIG. 15A;

FIG. 16A provides a table setting forth a set of ABATE air purification tests results of a fourth set of tests that were performed in accordance with the teachings of the disclosure;

FIG. 16B provides a graph pursuant to the tests results of FIG. 16A.

### DETAILED DESCRIPTION

In one aspect, disclosed herein are embodiments of devices, system, and methods for employing the same for the purpose of monitoring, measuring, testing, and reporting on the ability of Heating, Ventilation, & Air Conditioning (HVAC) and/or air purification (AP) systems to effectively deactivate airborne biologicals, remove dangerous volatile organic compounds (VOCs), and filter other harmful particulate matter out from the air. In various embodiments described herein below, the disclosed photocatalytic oxidization (PCO) based air purification devices disclosed herein were employed in a specially designed test system referred to as the Airborne Biological Assessment Threat Elimination (ABATE). As shown by the tests results presented below, the disclosed PCO-based purification units tested were shown to be able to clarify air traversing through ducting of the ABATE test system of at least 99.99% of particles and viruses.

Generally, a first system is provided wherein the system includes a uniquely designed intelligent HVAC unit that has been configured for not only providing fresh air to, and expelling unhealthy air from, a compartmentalized space, in this instance, a laboratory test environment, but doing so in conjunction with an air purification module that is capable of purifying the air of harmful compounds at typical HVAC operational parameters. Consequently, a second, test, system is also provided for safely determining the abilities of the presented HVAC and Air Purification systems for effectively purifying circulating air of harmful compounds.

Accordingly, in one aspect, provided herein is an efficient, intelligent air purification platform that employs photocatalytic oxidation in combination with air heating, cooling, and ventilation. Specifically, the present air purification systems may be combined with and/or be implemented within an HVAC unit, whereby UVC lamps and a metal mesh active matrix are used to produce a PCO reaction that functions to purify air as it traverses through the HVAC unit. In such an instance, the photocatalytic oxidation processes may primarily be accomplished by shining UVC light, for instance, in the 254-nanometer wavelength, on a Titanium Dioxide or zinc oxide coated mesh or matrix of metal. Also, UVC light produced by LED lamps may be used to create the catalyst, such in the 260-275 nm wavelengths, and filtration may also be achieved through use of a pre-filter, such as of MERV 11 to MERV 16 or higher HEPA filters. Post filter options may also be included, such as with one or more carbon and/or potassium permanganate filters.

In view of the above, the present intelligent HVAC system to be tested in accordance with the systems and methods herein disclosed may include one or more HVAC units, which may have on board processing along with intelligently controlled components including air-conditioning, compressor, condenser, valves, evaporator coil, air handler, thermal unit, chiller, and a ducting system for directing flow around the ducting as well as in and out of the air purification module. As indicated, one or more of these components may be intelligent, such as by including on-board processing, control, sensing, and communications units, such that the system 1 is capable of receiving and processing data to generate measurements of system performance.

Further, the present intelligent indoor air purification system to be tested in accordance with the systems and methods herein disclosed may include one or more of a PCO filter module, such as including an active PCO cell matrix that is configured for removing harmful and/or dangerous volatile organic compounds from the air by rendering them harmless and safe, such as by generating a photocatalytic oxidization reaction, whereby volatile organic compounds to harmless water and CO₂. The PCO filter module may include, in addition to the PCO cell matrix having a reactive mesh, e.g., a metal, such as a titanium dioxide or zinc oxide mesh matrix, one or more, e.g., a plurality, of lamps, such as UV lamps or UVC LED's. Together, these elements function collectively to remove harmful and dangerous volatile organic compounds (VOCs) from air traversing through the ducting of the test environment.

Specifically, VOCs are emitted gases from solids or liquids and have adverse health effects on humans. VOCs have higher concentrations indoors where they are trapped in the spaces they are emitting into. In various situations, these concentrations can be as much as 10 times higher inside than outdoors. This is very problematic since humans spend 90% plus of their time indoors. In fact, the EPA's Total Exposure Assessment Methodology (TEAM) studies have found VOC levels including about a dozen common organic pollutants to be 2 to 5 times higher inside homes than outside, regardless of whether the homes were located in rural or highly industrial areas. Additional TEAM studies indicate that while people are using products containing organic chemicals, they can expose themselves and others to very high pollutant levels, and elevated concentrations can persist in the air long after the activity is completed.

Previously, methods for treating air for VOCs have included the use of basic media filtration units, such as including MERV or HEPA filters that in and of themselves are not effective for removing VOCs. Hence, while media filters are a staple of filtration, and in various instances, effective against some particulates, they do not stop VOCs and/or gases, which easily pass right through such filters. The present PCO module, however, has been configured for efficiently and effectively purifying air of VOCs and harmful gases. Particularly, as determined herein, the PCO filter module is effective for deactivating biologicals, including bacteria, fungi, viruses, as well as eliminating volatile organic compounds (VOCs), such as paints, solvents, chemicals, and pesticides, and reduces odors, such as cigarette smoke and ash, garbage, animal odors, and the like. For instance, the present PCO filter modules evidence high level and broad-spectrum biocide efficacy, and are very effective at removing different types of VOC gases that are harmful to humans. For example, photocatalysis has broad-spectrum microbicidal activity that is effective for deactivating both enveloped and non-enveloped viruses.

In performing such PCO operations, the PCO module may use a mesh screen, such as a metal oxide, e.g., a TiO₂ or zinc oxide mesh, in addition to UV lamps or UVC LEDs, as reactants. Particularly, reactive TiO₂ has been found to be beneficial for use in treating air contaminated with VOCs. Specifically, a TiO₂ mesh framework can be employed in the PCO module as an environmentally friendly microbicidal agent for providing high level and broad-spectrum biocide efficacy during photocatalysis, which can be achieved without producing ozone.

Ozone, a lung irritant, is produced indirectly by ion generators, and other electronic air cleaners, and directly by ozone generators. While indirect ozone production is of concern, there is even greater concern with the direct, and purposeful introduction of a lung irritant into indoor air during a purification process. There is no difference between ozone outdoors, such as in smog, and ozone produced by ozone generation devices. Under certain use conditions ion generators and other ozone generating air cleaners can produce harmful levels of lung irritants.

However, although ozone is a harmful lung irritant, it does not actively attack biological tissues, as do the harmful viruses discussed herein above. These viruses not only impaired one's ability to breath they actively attacked the host organism, and in many instances, led to the death of the individual. Preventing the airborne transmission of such viruses, therefore, is of the utmost importance for preventing harmful injury and/or death that can be caused by breathing in such harmful elements, for instance, within a confined space. The air purification units disclosed herein have been developed to do just that. Likewise, the present ABATE testing system was developed to show that not only are the disclosed air purification systems capable of clarifying the air of harmful components, such as ozone, they are effective for deactivating airborne biologicals, such as with an effectiveness of 99.99%.

Particularly, as described in greater detail herein below, an important aspect of the testing performed shows that the ABATE system is effective at normal to high air flow velocities, regardless of virus type, and at increased viral load. For instance, air flow velocity is an important consideration not typically considered in prior iterations of legacy air purification systems. Specifically, in many early systems, UV light was thought to be the essential component for destroying reactive agents in the air.

Hence, in this regard, many prior systems would employ a single chamber having a tortuous air path through which air must travel as it traverses through the purification device. This winding air path was employed so as to slow down air as it passed through the device, thereby prolonging the exposure of the air to the UV light. Consequently, this air path would be replete with a multiplicity of UV light sources such that as the air was slowed within the chamber, the length of time any particulate or other volatile matter therein would then presumably be exposed to UV for a greater amount of time, thereby theoretically better effectuating protection. Hence, former systems were actually designed to slow down air speed velocity as a means of extending exposure to UV light.

However, contrary to these theories, the present PCO based air purification systems have been designed with an advanced configuration that recognizes that it is not so much direct exposure to UV light that affects virus viability, as it is exposure to a photocatalytic environment that produces significant viral deactivation. Thus, in various embodiments, the devices presented herein were designed to facilitate high speed air flow through the system, whereby one or more, e.g., two, three, four, five, or more photocatalytic oxidization chambers can be provided so as to deactivate viral loads. Consequently, the goal of the disinfection efficacy testing, set forth herein below, was to test the present device configurations with realistic airflows found in typical HVAC systems, which testing goes beyond any previous forms of testing that was available prior to the inventor's work in this field.

For instance, traditionally, virus testing in BSL Level 3 and Level 4 labs were conducted within a small ‘primary’ container that was worked on inside of a 6 foot or smaller bio-containment cabinet. A problem with this traditional setup, with respect to testing a bio-aerosol, such as Covid-19, is that it's not representative of the real-world conditions within which the virus would be aerosolized within a working or living environment, where air would be circulating throughout the environment, such as at increased velocities. Hence, such prior testing did not accurately account for how a virus, such as Covid-19, would realistically live and interact as it circulates through an HVAC system of a house or building.

Consequently, as set forth below, in various embodiments of the system, the ability to collect and measure viruses in an HVAC system with normal HVAC operating air velocities and CFM (cubic feet per minute) airflows was tested. For purposes of these embodiments, it was assumed that the collection and measurement was a standard air flow found within a traditional HVAC system such as 280-400 CFM per ton, as opposed to normal ambient room conditions. The tonnage was not as important as it can be scaled to any sized HVAC system on a 280-400 cfm per ton basis. 400 cfm/ton is a common standard for determining ‘standard air conditions and air volumes’ whereas 280 cfm is a typical level found reflecting less than ideal ventilation air flow. The primary purpose of these embodiments was to collect and measure the viruses for airborne transmission research.

Traditionally, collecting viruses and measuring them has been extremely challenging from both a bio-safety and measurement perspective. With respect to measuring the viruses and particulate data, one challenge is that most particulate metering devices can only measure at 1 to 3.5 cfm or 100 liters/minute or less. The present embodiments overcome these challenges with deploying custom shrouded isokinetic sample probes, described below, which allow the air aerosolized with viruses or bacteria to pass through but at substantially slower air velocities, while at the same time as allowing air of higher flow velocity to pass right on by without affecting the measurements to be taken.

These custom shrouded probes slow the captured air down to under 3.5 CFM so that the particulate sensors can measure the air and with a separate shrouded tube for virus capture, can capture any aerosolized virus for determining pfu's (plaque forming units), qPCR (Quantitative polymerase chain reaction) detection, TCID₅₀, and/or for performing other virus measurement assays. Another unique feature of the shrouded probes are their adaptability for use in conjunction with a specially designed collection chamber that allows the air to pass through the collection device and to then to be fed back into the duct system.

Another benefit of the ABATE test system provided herein is its modular configurability. For example, if the infectious virus to be tested is dangerous, it is required to be tested and measured in a biosafety lab of either level 2, 3, or 4. The more dangerous the virus to humans, the higher the level of lab required to contain and conduct research on it. Further, dependent on how dangerous the biological is, it will also require the system to be air-tight and contain the aerosolized virus becoming the primary container. For these reasons, most biosafety labs, specially at levels 3 and 4 are relatively compact in size. Consequently, the modular design of the ABATE test system allows it to be expanded in larger environments, but may also be configured to be more compact, such as for use in testing in smaller lab environments. This is especially useful for testing involving the novel Coronavirus SARS-CoV2 (COVID-19), which currently requires a Level 3 bio-safety lab.

For these purposes, a special closed system embodiment of the ABATE system, as described below, was used to collect and measure SARS-CoV2 while proving the air purification of the system was capable of deactivating the virus. In this embodiment, two shrouded sample tubes were used to measure particulates at the point of nebulizing and at the point of virus collection. These particulate measurement sensors showed that they were capable of measuring down to 0.3 microns and collected statistics which were binned and collected together in 0.3-0.5 micron, 1.0 micron, 2.5 micron, 4 micron and 10 micron size categories. Additional particulate mass numbers and average sizes were also collected. In such instances, all particulate data can be collected in 1 second increments and can be measured over the multi-pass or single pass duration term.

A third shrouded, viral, collection probe can also be used, e.g., at the point of virus collection, such as for sampling the virus for qPCR analysis. In this instance, the shrouded tube was connected to a 47 mm filter chamber with a PTFE 47 mm hydrophobic 0.2 micron filter for collection. A different sized filter may also be utilized. Further, different mediums besides the PTFE can be tested and can also be used including Polycarbonate, Polyester and Polyethersulfone of both hydrophobic and hydrophilic properties and different micron sizes. In various instances, two PTFE filters for duplicate testing can also be used by being positioned directly within the HVAC ducting system post filtration to collect virus sampling for CPE analysis. An internal reducing transition duct can further be used to focus the air for capture. Different sized transition ducts can be used depending on conditions desired.

The referenced special closed system embodiment allowed for testing of all of the various embodiments of air purification, which included single, double, triple and quadruple PCO chamber configurations, and with and without MERV filter media. These parameters were also employed for testing the portable and HDAZ engine-based product versions, which were tested in a multi-pass configuration. A single pass version of the system could also be used, which would allow for breaking the system loop and allowing air intake from the lab and exhaust either through a direct connection to the lab exhaust or via exhausting through a HEPA filter into the negative pressure BSL Level-3 lab room, which may then be exhausted through the lab room's additional HEPA filter. In any of these instances, these systems can be used to test and measure a multitude of biologicals and different aerosolized conditions. Accordingly, in various embodiments, the HVAC system and unit may include one or more particulate probes, such as positioned at the beginning, e.g., opening, of the HVAC unit and/or positioned at the egress or exit of the HVAC unit. The unit may also include a virus collection and sample location and/or filter. A plurality of filtration units may also be provided, such as in line with the air flow. One or more nebulizers may also be included.

Specifically, the present testing, set forth herein below, was performed to determine whether Covid-19, and its variants, could survive in HVAC systems, and as a result of determining that it could, the present devices, their configurations, and the attendant systems were developed and deployed in methods for deactivating such viruses. In these regards, various iterations of the air purification module were tested in the in-duct ABATE system presented herein and through this testing it was determined that the presented air purification devices were found to be capable of deactivating Covid-19 present in the air traversing in an HVAC system. More specifically, three different versions of the present air purification system, including a large industrial use, in-room portable, as well as a small automotive/aviation version were tested against several different variants of SARS-CoV2, including: Beta, BA.2, Omicron and Delta variants, and well as RSV and the Flu. Through these studies it was found that the present devices, systems, and their methods of use were effective for reducing, e.g., deactivating, all of the above referenced viruses tested as well as bacteria, microbes, pollen, dust, dander, allergens, spores, fungi, mold, odors, smoke, and toxic fumes, without producing any harmful ozone or biproducts.

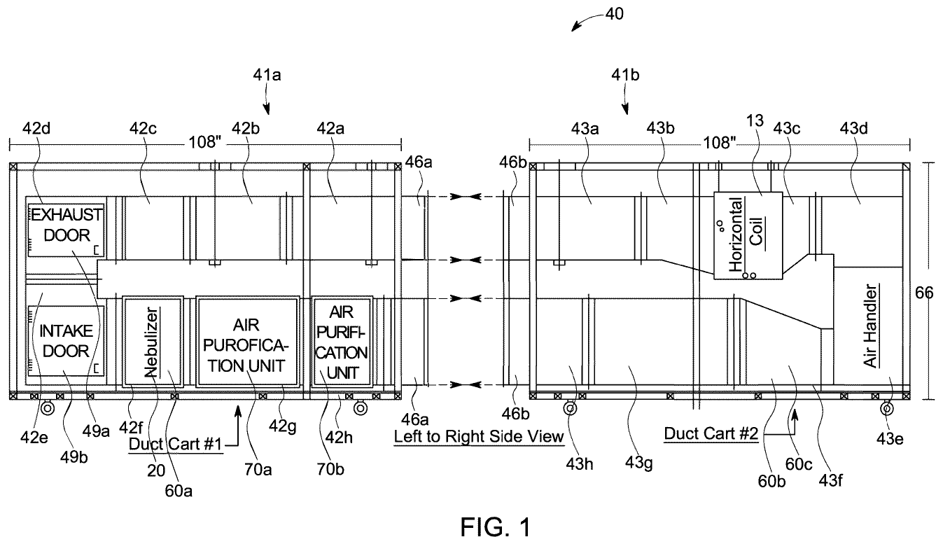

Accordingly, in view of the above, and as can be seen with respect to FIG. 1, provided herein is an airborne biological assessment threat elimination (ABATE) system 1 for collecting and measuring airborne biologicals. Particularly, there are two key aspects to this ABATE system 1. The first is an indoor air purification system 70, such as presented in U.S. Pat. Nos. 11,635,221 and 12,111,064, the subject matter of which is incorporated herein by reference in its entirety for all purposes. The second key aspect is the ABATE testing architecture 40, as presented herein, which may be employed in a confined, e.g., laboratory, environment 95, wherein the referenced air purification systems of the '221 and '064 patents can be tested in a manner that simulates the typical airflow through a highly populated building or facility.

In such instances, the air flow may be from about 1,000 Cubic Feet per Minute (CFM) or less to about 10,000 CFM or more, such as about 2,000 to about 8,000 CFM or above, for instance, from about 3,000 CFM to about 6,000 CFM, including about 4,000 to about 5,000 CFM, e.g., up to and above 50 MPH, which is an air velocity of at least 4× greater than the velocity of air flow passing through a typical commercial HVAC system. In this regard, the ABATE system can be configured to efficiently and effectively handle from about 2 to 4 air changes per hour up to and above 60 air changes per hour (ACH), such as about 5 or 10 to about 40 or 50 ACH, for instance, about 15 or 20 to about 30 or 40 ACH, including about 25 ACH. The ability for the herein presented ABATE system 1 to meet and exceed these standards is important for verifying the usefulness of the indoor air purification system 70 to be able to deactivate airborne biological threats, such as by clarifying the air of viruses, other biologicals, Volatile Organic Compounds (VOCs), and other harmful particulate matter at typical HVAC running conditions.

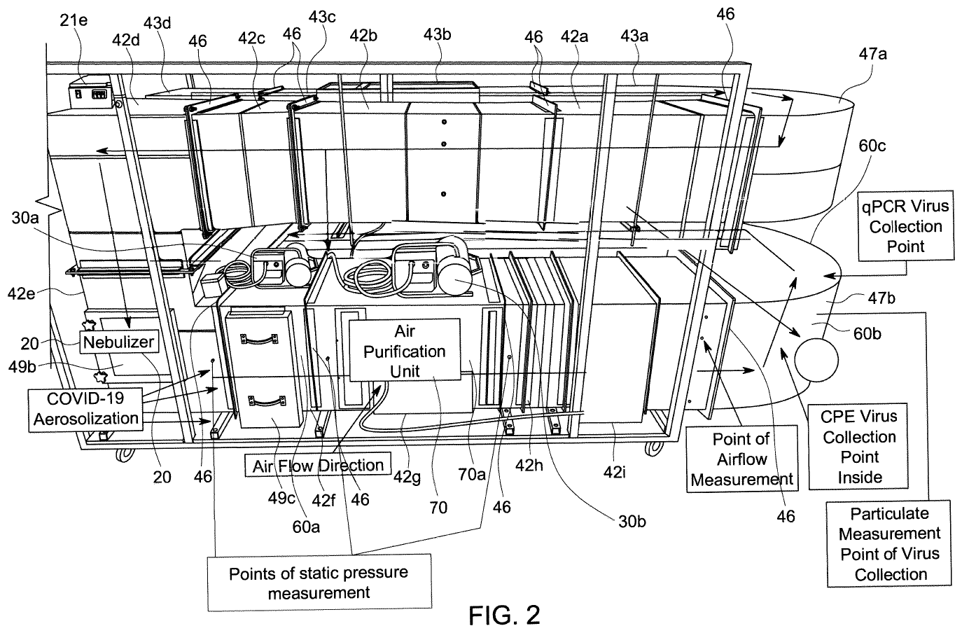

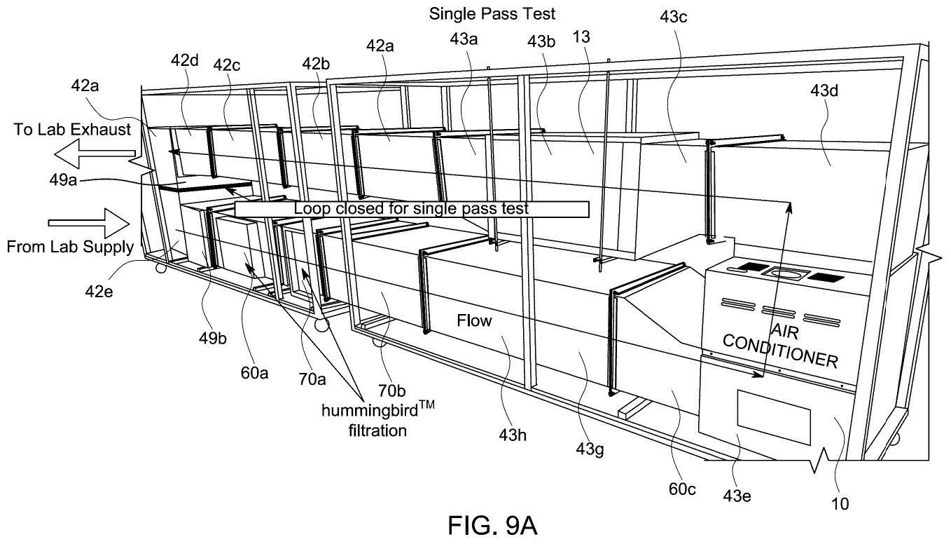

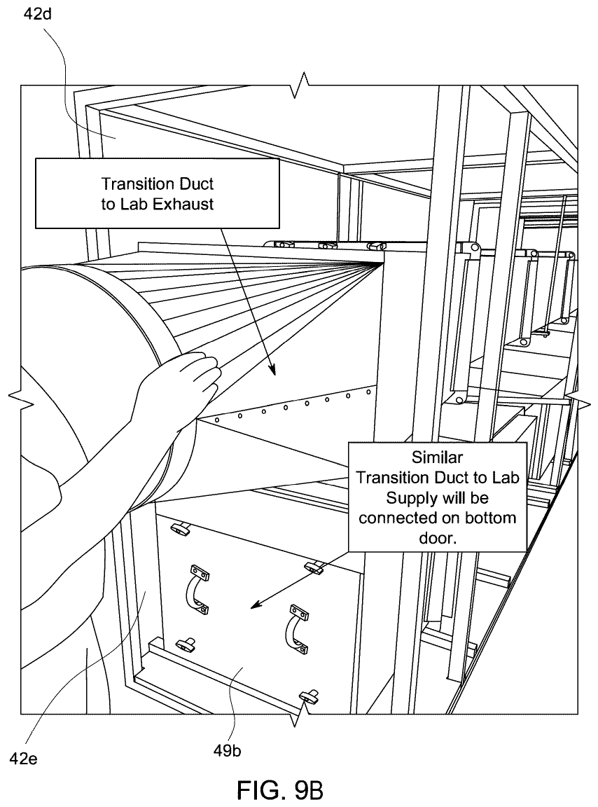

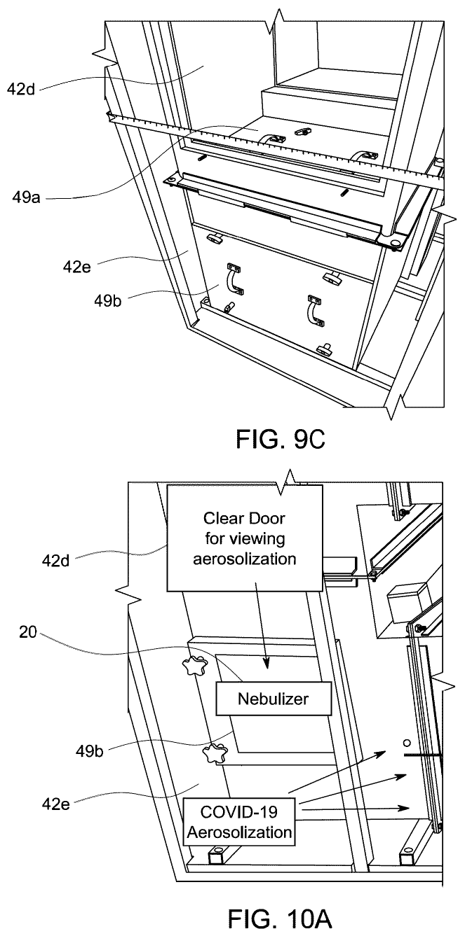

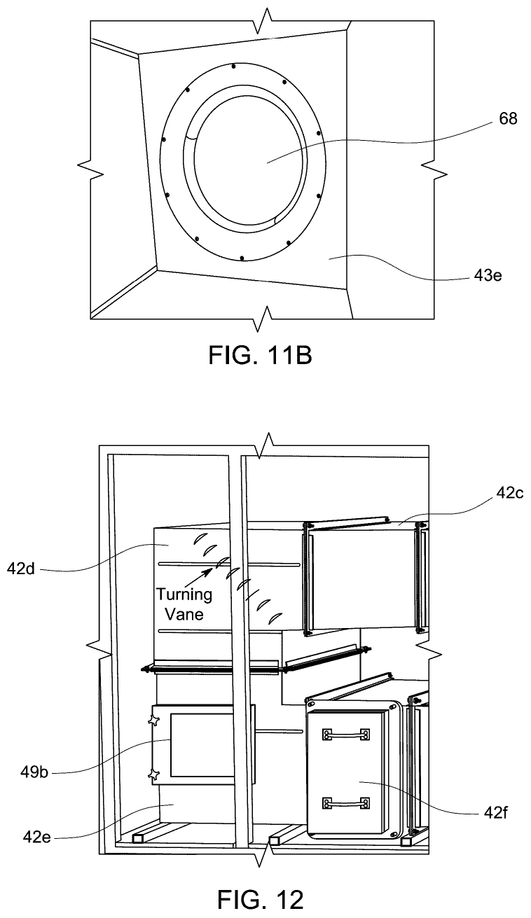

Consequently, to adequately show the efficiency and effectiveness for an air purification system 70 to clear the air passing through a Heating, Ventilation, and Air Conditioning (HVAC) system 10, the ABATE architecture 40 may be configured for including an entire HVAC system 10 or a portion thereof. For example, as presented with respect to FIGS. 1 and 2, the HVAC system 10, in these instances, primarily includes an air handler 15, for pushing air through the various ducting modules 41 of the system 1, as well as a coil 13, such as one or more of an evaporator and/or condenser coil, which may include a refrigerant that is adapted for removing heat from the air flow, thereby cooling it. However, in other embodiments, such as with respect to FIG. 9A a more complete air conditioning unit 10 may be included, such as including one or more of: an air conditioner, a compressor, a condenser coil, a thermal expansion valve, and an air handler. In a further instance, as depicted in FIG. 11A, a commercial sized industrial fan 68 may replace the air conditioning unit 10 of FIG. 9A, such as for circulating an airflow through the ducting system 40, such as at higher airflow velocities.

Accordingly, in its simple form, the ABATE system 1 may merely include an air handler 15 for circulating airflow within the ducting system 40, and may further include a condenser coil 13 for conditioning that airflow. However, in a more complex embodiment, the ABATE system 1 may include a full air conditioning unit 10 having its own air handler 15 for circulating are throughout the ducting 40, and may further have its own refrigerant containing coils 13 for conditioning the air as it passes through the air conditioner 10. In such an instance, the air conditioner 10 may be positioned within, or may otherwise be coupled with, the ducting sub-system 40. Dependent on the test parameters, a suitable air conditioner 10 can be any unit that is configured for circulating airflow and/or removing heat from the air traversing through the ducting subsystem 40.

For example, any mechanism that is capable of circulating air and/or for transferring heat from within the environment of the ducting and into a heat capturing medium, such as refrigerant, may be employed. In such a process, the heat capturing medium, e.g., refrigerant, may be contained within an enclosure within the air conditioner 10, and may be configured for being converted from a cooled refrigerant to a warmed refrigerant, thereby cooling the interior environment within the ducting. Further, in particular embodiments, a furnace can also be included so as to warm the air traversing through the ducting. In particular embodiments, none of an air conditioner 10, air handler 15, or condenser coil 13 need be provided. Rather, an industrial sized fan 86 may be substituted for these elements. See FIG. 11B.

However, where an air conditioning unit 10 is provided, one or more compressors can also be included. For instance, a compressor can be coupled to one or more of the refrigerant coils, e.g., copper coils, of the air conditioner 10. Particularly, the compressor can be configured for compressing the warmed refrigerant into a warm pressurized refrigerant, whereby once the warmed refrigerant is pressurized, the compressor can then move it throughout the copper coils.

Likewise, a condenser coil can be provided and can be coupled to one or more of the copper coils and the compressor. In such instances, the condenser can be configured for removing the heat from the warm pressurized refrigerant so as to produce a cooled pressurized refrigerant. A thermal expansion valve can also be included and can be coupled to the compressor, such that when the valve is open, the built-up pressure is released from the copper coils thereby converting the cooled pressurized refrigerant into a cooled refrigerant capable of being reused to further cool down the air flowing through the ducts, such as in a multi-pass configuration.

Accordingly, in various embodiments, an air handler 15 may be provided such as by being coupled to, or otherwise be inserted within, the ducting elements 43 of the ABATE system 1. In such instances, the air handler 15 may be configured for pushing air throughout one or more of the ducting subsystems 40. However, in various other embodiments, the ABATE system 1 may include an air conditioner 10. In such an instance, the air conditioner 10 may itself include an air handler 15, such as where the air handler 15 of the air conditioner 10 is configured for circulating air through the ducting modules 41 of the ABATE system.

In such embodiments, the air handler 15 may be configured for further taking air, e.g., warm air, in from an outside environment, e.g., laboratory intake, and pushing it through the various components of the HVAC system 10, where that air can be cooled. In either instance, the ABATE system 1 may include air handler 15 that is configured to push air, such as air containing a nebulized and/or aerosolized airborne biological, e.g., virus, through the enclosed ducting system 40, where that air may then be pushed through an air purification system 70, so that the airborne virus can be deactivated thereby. Accordingly, an important aspect of the present innovations is the proprietary air purification module 70, which was the subject of the ABATE testing set forth herein, as described in detail below.

Specifically, in one aspect, the herein presented ABATE system 1 may include an air purification module 70 that contains a photocatalytic oxidization air purification unit 75. As can be seen with respect to FIGS. 1 and 2, the air purification module 70 may be communicably coupled to an air handler 15 or an HVAC subsystem 10, such that as air is circulated by the air conditioner 10 and/or air handler 15 that air is pushed through the air purification module 70. For instance, in particular embodiments, the air purification module 70 may be placed within, or in substitution for, one or more duct elements 42 of the ducting system 40 in such a manner that it is in line with the air conditioner 10 and/or air handler 15.

In such instances, the air conditioner 10 and/or air handler 15 may also be contained within, or otherwise substituted for, one or more of the duct elements 43. As explained in detail below, once so positioned, any viruses present within the air circulating through the photocatalytic oxidization unit 75 will be deactivated and any VOCs therein will be decomposed. For these purposes, the air purification module 70 may include a photocatalytic oxidization unit 75 having one or more treatment chambers that define one or more treatment zones.

In this regard, each treatment zone of the photocatalytic oxidization chamber 75 of the air purification unit 70 may be defined by one or more sets of UVC lights. These sets of UVC lights may be positioned within the chamber so as to be in an offset configuration from one another. For example, in various embodiments, a plurality of rows of offset panels of UVC lights may be provided, whereby the rows of UVC lights are separated from one another so as to form a space therebetween. This space may include a photocatalytic oxidization cell panel positioned therein and proximate each of the UVC light panels. In such a configuration, the space between the UVC lights may form a treatment zone, such as where the panels of UVC lights define the bounds of the treatment zone, and the placement of the photocatalytic oxidization cell panel within the treatment zone allows a photocatalytic oxidization reaction to be produced within the chamber when the UVC lights shine on the photocatalytic oxidization cell panel.

Specifically, the photocatalytic oxidization cell panel may be formed as a metal dioxide mesh grill such that as the UV lights from the UV light sources, e.g., UV LEDs, shine on the metal dioxide cell panel, a photocatalytic reaction is thereby produced within the chamber. In this manner, the results of the UVC light interacting with the metal dioxide, e.g., titanium dioxide, is the production of a photocatalytic oxidization reaction that generates a number of reactive products that are then contained within the reaction chamber. Hence, as the air flows within and through the treatment zone, the generated reactive products within the chamber interact with any viruses present within the airflow thereby deactivating them, and further decompose any volatile organic compounds converting them into inert compounds, such as CO₂ and H₂O.

In this manner the air traversing through the air purification module may be cleared of volatile organic compounds, while at the same time any viruses therein may be deactivated. A unique advantage of the present air purification module 70 is that it is capable of deactivating airborne biologicals, while also clarifying the air of volatile organic compounds VOCs), all while the air is flowing throughout the ducting 40 at normal to high airflow velocities as would be utilized by a residence, office building, or large industrial complex. In such instances, it is important that the ducting sub-system 40 is air-tight and leak proof, such as within the testing environment 95, so as to prevent the airborne biological from escaping from the ducting 40 at high airflow rates.

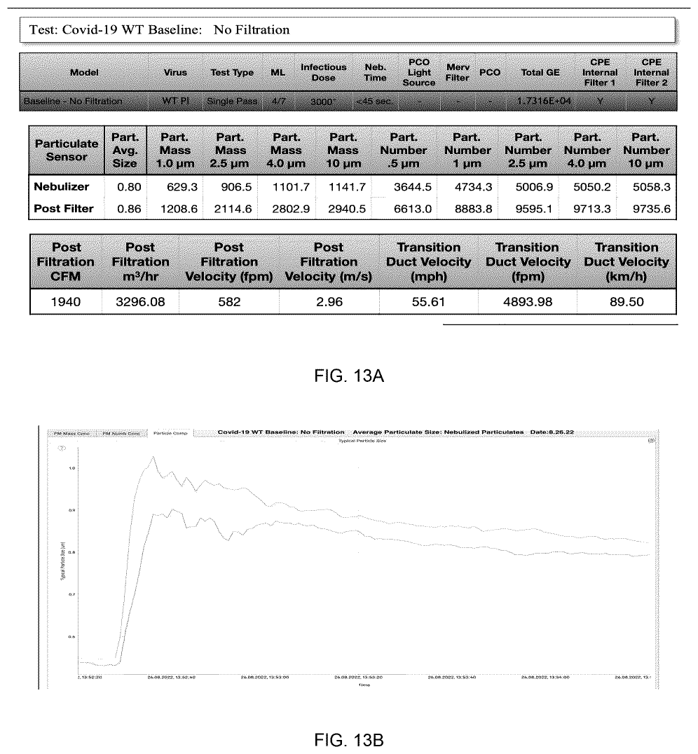

For example, in various embodiments, as shown in FIG. 11B, the air conditioner 10 and/or handler 15 may be replaced or augmented by including a fan unit 68 for pushing the requisite air throughout the ducting subsystem 40. Particularly, as discussed herein below, in order to simulate a common sized HVAC system, handling air at a typical flow rate of 500 to 1000 Cubic Feet per Minute (CFM), the initial ABATE system was configured with a 5-ton air handler 15 and furnace, along with a condenser coil 13, and up to about 25 or up to 50 feet or more of ducting 40. As can be seen with respect to FIG. 13A, this initial system was tested so as to determine airflow through the ducting system 40, but without the presence of an air purification system 70. Thus, this configuration of the ABATE test system 1 acted as a control by which the other air purification system configurations could be compared.

In these regards, with reference to FIG. 1, this control ABATE system configuration only included an HVAC Unit, which was initially employed with only air handler 15 (or fan 68), but without the addition of the air purification unit 70. In this particular regard, as described in greater detail herein below with reference to the experiments performed herein, this version of the system, without the purification unit 70, was employed as a control test to verify the fact that both biologicals, e.g., viruses, and volatile organic compounds (VOCs) could be aerosolized and circulated throughout the air traversing through the HVAC 10 and its associated ducting 40, without being completely deactivated. This was found to be true, as shown below. It is to be noted that these initial tests were performed with both a MERVE and a HEPA filter, and regardless of whether a MERV, or even a HEPA, filter was included live, airborne virus was detected in the air flow.

However, because live virus was being tested, the present ABATE architecture 40 required a specially designed ducting subsystem. Consequently, another aspect of the innovations provided herein is the unique ducting subsystem 40. As can be seen with respect to FIGS. 1 and 2, the ducting subsystem 40 may be a modular system, such as including one or more, e.g., a plurality, of modules 41, where each module 41 is composed of a number of duct elements 42, 43. For instance, as can be seen with reference to FIGS. 1 and 2, two different configurations for the modular ducting subsystem 40 of the ABATE testing system 1 are presented. For the most part, the two different configurations largely include the same system components, but differ in their arrangement of ducting elements 42, 43. For example, the ABATE system 1 presented in FIG. 1 is configured as a long, expanded and stacked testing architecture, such as composed of two stacked modules 41a and 41b, such as where each module is about 25 feet in length. This would be useful where the lab environment 95 is expansive, and can accommodate about 50 feet of ducting 40. The ABATE system 1 of FIG. 2 is in a retracted, back-to-back, stacked configuration, which basically takes up only half of the space as the configuration of FIG. 1.

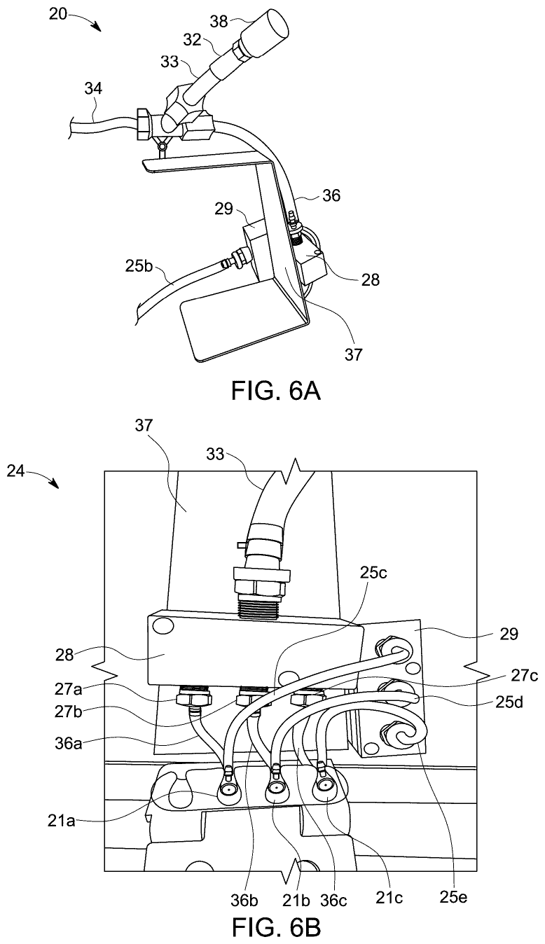

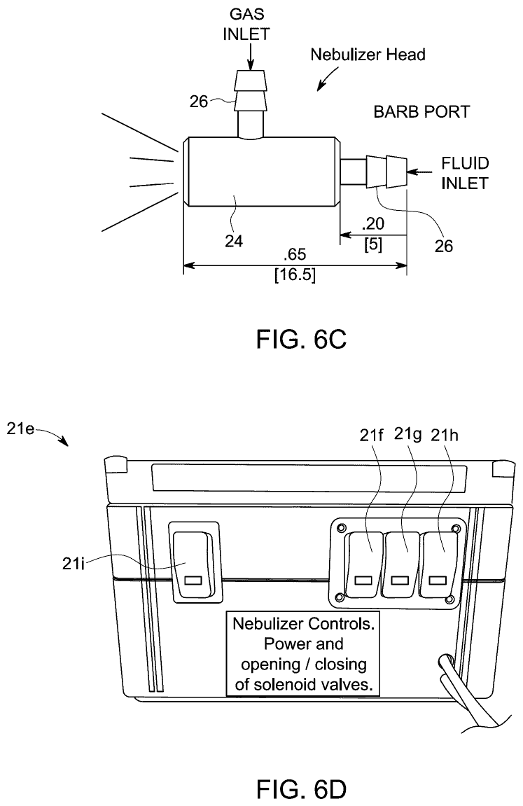

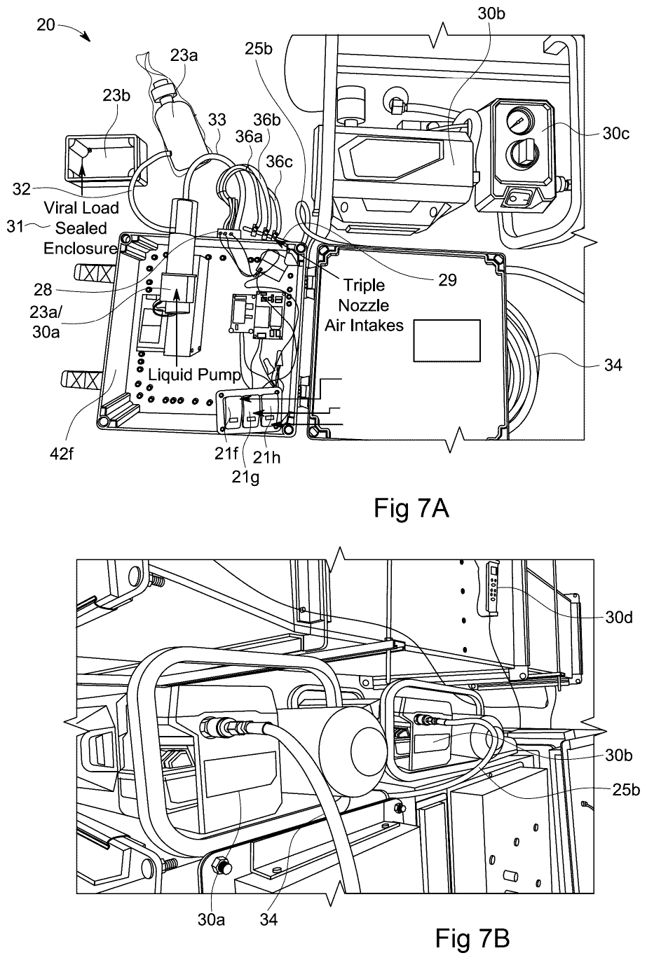

In either configuration, the ABATE system 1 may include the same basic components. For example, as can be seen with respect to FIGS. 6 and 7, the ABATE system 1 may include a nebulization and/or aerosolization unit 20, for converting a biological, such as a virus, from a solid, e.g., powdered, or fluid form, into an airborne form that can be carried in the air through the ducting subsystem 40 and into the air purification module 70, whereby the airborne biological, e.g., virus, can be deactivated. In its basic form, the nebulization unit 20 may include a plurality of chambers, such as a fluid chamber 23a including a fluid such as saliva, mucus, or a synthetic form thereof, and a particulate matter, e.g., viral load, chamber 23b.

A number of translation conduits 32, 33, 36 may also be included, such as for allowing the two compositions to be combined, to interact, and/or to mix, such as in a third, mixing, chamber 28, e.g., within a liquid manifold. In certain embodiments, a fluid pump 23 (or compressor 30a) may also be included, such as for effectuating the translation of the fluid, e.g., saliva or mucus, from the fluid chamber 23a, and/or the biological load, from the load chamber 23b, via respective translation conduits 33 and/or 36 for mixing and/or translation to the mixing chamber 28. Once in the mixing chamber of the liquid manifold 28, the liquefied load, e.g., virus, containing composition can be conveyed into a nebulization chamber 24 along with pressurized air, e.g., from an associated air line manifold 29, in such a manner that within the nebulization chamber 24, the mixture of saliva (or mucus) and the virus can be nebulized and/or aerosolized, as shown in FIG. 6C. This aerosolized mixture can then be transmitted to the sample collection duct element, in this instance 42g, for testing, such as depicted in FIG. 5.

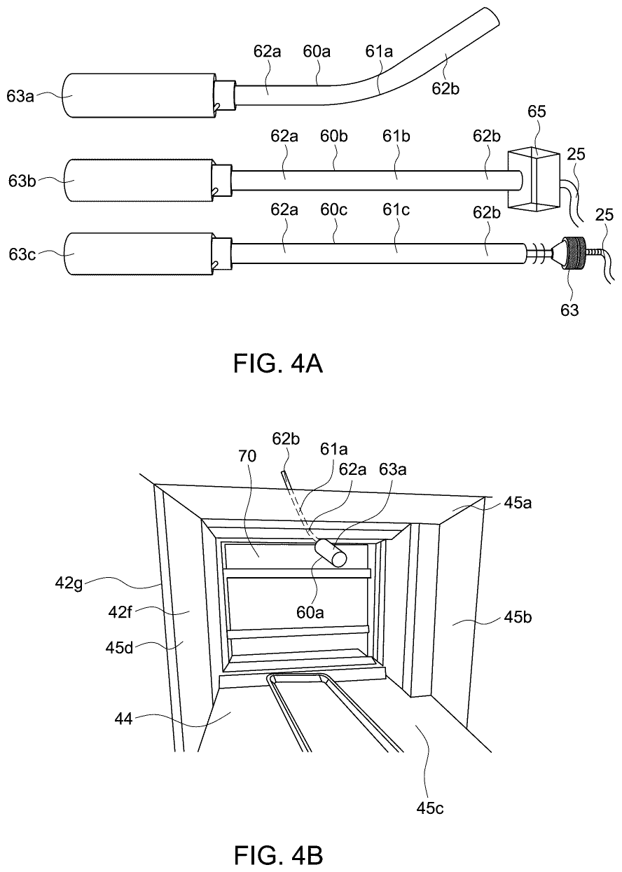

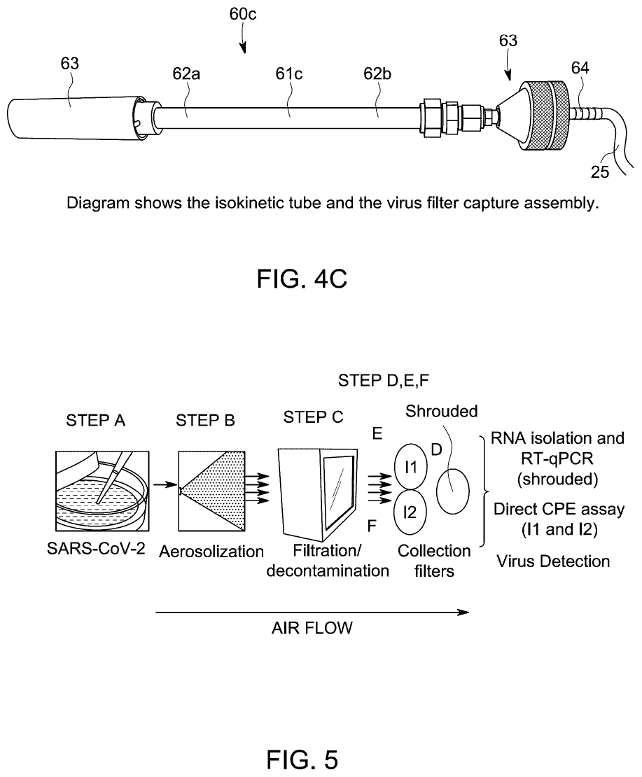

Particularly, an aspect of the ABATE system 1 is that it is designed for testing the ability of the air purification module 70 to deactivate air borne biologicals, such as viruses, as well as to remove volatile organic compounds, and other particulate matter, from the air of a laboratory environment 95, while the air is flowing at a relatively high velocity. For these purposes, the ABATE system 1 may include one or more sample collection probes 60, which can be used to determine a quantity of particulate matter being transported within an airflow. More specifically, as can be seen with respect to FIG. 4A, in another aspect, provided herein is a unique sample collection probe 60. The sample collection probe 60 is unique because it is configured for collecting particulate matter from out of the air, while at the same time allowing the air to continue to flow through and around the collection probe 60 so as to be returned to the ducting system 40 after measurement.

For instance, in one embodiment, a first sampling probe 60a, e.g., a particulate matter probe, may be provided where the sample probe 60a is at least partially contained within a ducting element 42f of a ducting module 41. Since the sample collection probe 60a is positioned within the duct element 42f, this duct element may be referenced herein as a sample collection duct 42f of the first ducting module 41a. As can be seen with respect to FIG. 4A, in various embodiments, one, two, three, or more sample collection probes 60 may be included, such as within one or more duct elements 41. More particularly, as can be seen with respect to FIG. 4B, the first sample collection probe 60a is positioned within the first sample collection duct element 42f of the first ducting module 41a for collecting a nebulized and/or aerosolized sample upon initial nebulization and delivery into the ABATE system 1, e.g., in advance of the air purification module 70.

More specifically, the first particulate matter sample probe 60a may be positioned in a sample collection duct element 42f in advance of an air purification module 70 positioned in an adjoining purification duct element 42g. The particulate matter sample probe 60a may be composed of an elongated reception tube 61a, which may be configured on one end 62a for collecting air containing particulate matter within the collection tube 61a, and on the other end 62b for allowing the collection and measurement of that particulate matter. For instance, as can be seen with respect to FIG. 4B, the sample collection duct element 42f may be composed of a plurality of walls 45a, 45b, 45c, and 45d, wherein the walls are formed into opposed pairs so as to form the shape of a cube having a cavity 44 being defined by the pair of opposed walls 45. Consequently, for measuring the particulate matter that gets nebulized and/or aerosolized and introduced into the ducting system 40, the particulate matter sample probe 60a may be situated within the cavity 44 of the sample collection duct element 42f so that the portion 62a of the probe that collects particulate matter is within the cavity 44 and subjected to the airflow, while the portion 62b of the probe where measurements are performed 65 may be positioned outside of the cavity 44, and thus, will not be subjected to the flow of air.

For these purposes, the elongated reception and collection tube 61a may include a first portion 62a and a second portion 62b, where the second portion 62b is angled or curved with respect to the first portion 62a. This curvature is useful because it allows the first portion 62a to be retained within the cavity 44 of the duct element 42f, so as to collect particulate matter, while allowing the second portion 62b to extend outward through one of the walls 45 of the duct element 42f so as to be accessible to the outside. This configuration is useful because it allows a separate independent particulate measurement device 65, for measuring collected particulate matter, to be positioned outside of the duct element 42f so as to take measurements in a manner that the flow of air does not interfere with the measurement of the particulate matter.

Historically, such measurements were difficult to take because the speed of the airflow causes interference with the taking of the measurements. This interference is not unsubstantial, and therefore, specially designed sample probes had to be engineered. Specifically, as can be seen with reference to FIG. 4A, the collection end of the sample probes 60 have been fitted with independent shroud members 63 that have been specifically designed to slow the air entering the elongated reception tube 61. In this way, as the air traverses the shroud 63, a tortuous flow path is created such that by the time the air traverses to the chamber of the particulate measurement unit 65, the air has been sufficiently slowed so as to allow the measurement of a sample of particulates being transmitted within that flow of air, in a manner that the flow itself does not affect the measurement. This configuration is useful because typical measurement devices are basically inoperable at the air flow speeds that would typically be required for conditioning air in a home or commercial space, such as at 1000 CFM to 2000 CFM, or even up to 3000 CFM or 5000 CFM or more. At such air flow speeds, the actual flow of the air becomes an impediment to measurement. Thus, such testing instrumentations and methodologies for implementing them, as presented herein, have opened up a brand-new field for testing aerosolized biomatter traversing through an HVAC system at typical operational parameters.

Likewise, as can be seen with respect to FIG. 2, a second, post filtration sample collection probe 60b may be included within a second sample collection duct element, in this instance 47b, of a second ducting module 41b. Particularly, a second sample collection probe 60b may be included and positioned in the second sample collection duct element 47b, in a manner similar to that of the pre-filtration sample collection probe 60a. For example, the post filtration particulate matter sample probe 60b may be situated within the cavity 44 of the sample collection duct element 47b so that the shrouded portion 63b of the elongated reception tube 61b of the probe 60b, which collects particulate matter, is entirely within the cavity 44 and is subjected to the airflow.

However, the portion of the reception tube 62b, where measurements are performed, is positioned outside of the cavity 44, and thus, is not subjected to the flow of air. In this instance, because the sample collection duct 47b is curved, the elongated reception tube 61b of the post filtration sample probe 60b need not be curved, although it can be if desired. In either instance, where the portion of the elongated reception tube 60b transitions through a wall 45 of the duct element 42, one or more air and/or waterproof sealing members, such as a gasket, O-ring, and the like, may be included, for instance, positioned on either side of the wall, so as to make the transition seal proof.

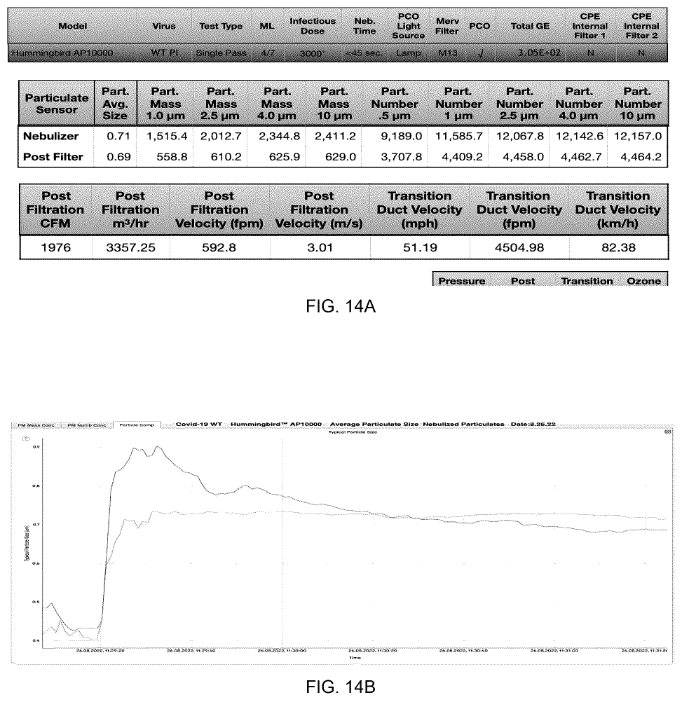

In view of the foregoing, the ABATE system 1 may be configured for nebulizing or otherwise aerosolizing particulate matter, such as including a biological, e.g., a viral load, and circulating that aerosolized virus through an air purification system 70, such as for testing the ability of the air purification system 70 to deactivate that airborne virus. A key feature of the ABATE system 1 is that it includes an HVAC unit that is adapted to function as a typical HVAC system would function when servicing a home, commercial building, and/or an industrial complex. However, as shown in FIGS. 14, 15, and 16, the present ABATE system 1 can be configured for testing whether a given air purification module 70 is capable of clarifying the air circulating through an HVAC system, such as by de-activating the virus therein, as the circulated air is pushed through the air purification system 70 at higher air flow velocities, e.g., of about 1500, 1800, or even 2000 CFM or more.

For example, as can be seen with respect to FIG. 5, a methodology for testing the effectiveness of an air purification module 70 is presented. In accordance with the methodologies described herein, the first and second Steps A and B, are to produce, e.g., grow, the biological to be tested, and then to nebulize or otherwise aerosolize that biological so as to produce an air borne, e.g., aerosolized, biological that is capable of being circulated through an HVAC, e.g., test, system 10. This process of nebulization is described in greater detail herein below with reference to FIGS. 6 and 7.

However, in accordance with Step C of FIG. 5, once the biological is aerosolized, it can be delivered into the ducting 40 of the ABATE system 1, so as to be circulated therethrough and subjected to purification. Specifically, once the biological is aerosolized, it may then be flowed through the air into the air purification system 70. While in the air purification unit, the air can be purified, and presumably, the biological can be deactivated. A key component of this process, however, is determining how much of the biological is aerosolized and transmitted through the air purification module 70. So, being, in accordance with FIG. 4, it is useful to not only include a particulate matter collection and measurement probe 60a at a pre-filtration position, but it is also useful to place a further particulate matter collection and measurement probe 60b at a post-filtration position as well. Hence, a first probe 60a may be positioned (at least partially) within a pre-filtration duct element 42, so as to be positioned proximate the point whereby the aerosolized biological is introduced into the ducting 40, while a second probe 60b may be positioned (at least partially) within a post-filtration duct element 43.

Consequently, as the aerosolized biological is introduced into the nebulization duct element 42, it may be measured, such as in a pre-filtration particulate measurement process. Likewise, as the aerosolized biological exits from the air purification unit 70, it may be measured again, such as in a post-filtration particulate measurement process. Such measurements are useful because an important aspect of the purification devices 70 herein disclosed is that they are capable of purifying the air at typical to high air speed velocities. For instance, as can be seen with respect to FIG. 1 or 2, in one embodiment, once introduced into the ducting 40, the aerosolized biological can be circulated through the ducting 40 via the air handler 15. However, in another embodiment, as set forth with respect to FIG. 11A, the aerosolized biological may be circulated through the ducting 40 via an air conditioning unit 10. This latter iteration is useful when performing the tests described herein, such as when the system is set up to be run under conditions similar to those where an HVAC 10 is configured to service a house or small business.

For example, the air flow velocity within the ducting 40, produced by an air handler 15, may be upwards of 1000 to 1500 CFM, while the air flow velocity produced by an air conditioner 15 may be upwards of 1700 or 1800 CFM. However, sometimes it is useful for performing tests where the air flow velocity is up to and above 2000 CFM. In this instance, therefore, as can be seen with reference to FIG. 11B, in various instances, it may be useful to circulate the air through the ducting at much higher air flow velocities, such as at air speed velocities upwards of 1700 or 2000 or more CFM, such as more than 3000, 4000, or upwards of 5000 or even 10,000 CFM. For such purposes, an industrial fan 68 may be employed so as to generate the desired air flow velocities. However, in such instances, the air conditioning unit 10 of FIG. 11A can be replaced by the fan unit 68 of FIG. 11B.

Particularly, as set forth with regard to the testing described below, the ABATE system was tested up to 2,000 CFM, which is a realistic airflow level for an air conditioning unit servicing a large home or a standard commercial facility. However, as indicated, in initial testing, such as with the air conditioning configuration of FIG. 11A, it was determined that the configuration of the pre-filtration ducting elements 42, which were adapted to focus the airborne biological, e.g., virus, along the ducting and into the air purification unit 70, caused the static pressure within the ducting system 40 to slow the air flow to ranges from 700-1000 CFM. While this airflow velocity is sufficient to service a reasonably sized residence, it would not be sufficient to provide air conditioning for larger commercial buildings and complexes.

Hence, as can be seen with respect to FIG. 11B, to overcome the effects of this internal static pressure on air flow, and to increase airflow capacity, a large commercial fan 68 was added to the ducting subsystem 40 to achieve a target CFM rate of 2,000 CFM for in-duct testing. This 2000 CFM airflow velocity is the type of air flow that would be useful for servicing a much larger facility, and thus, testing the ability of the air purification module 70 to clear the air of viruses in larger facilities, at greater airflow velocities, was better shown in this configuration. However, it is noted in this regard that static pressure within the ducting system 40 is a useful variable to account for when determining the ability of the air purification module 70 to purify air at normal operating conditions. This is because the greater the static pressure is within the system 1, the slower the airflow will be, and the less likely the testing will be to accurately reflect normal operating conditions of HVAC systems.

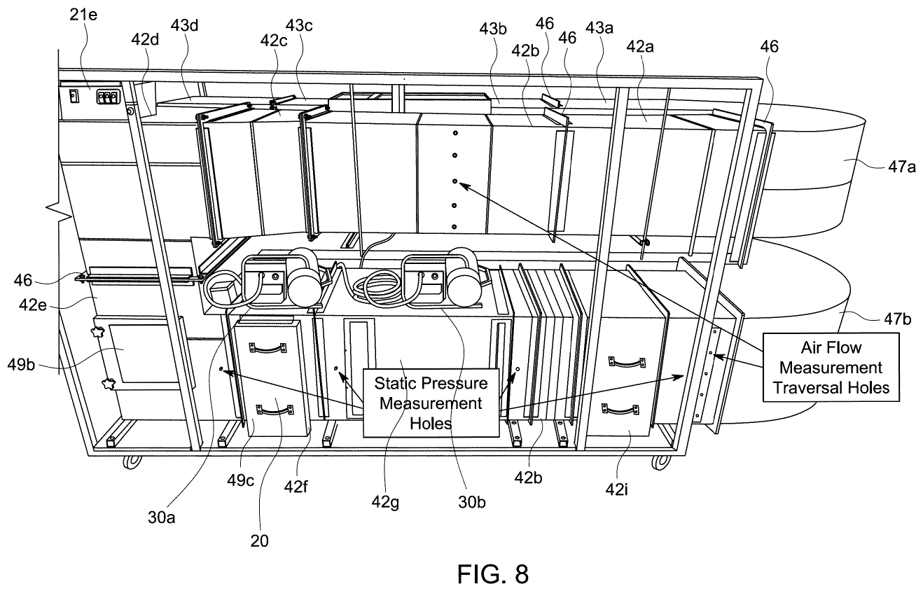

Consequently, as can be seen with respect to FIG. 8, in various embodiments, the ducting subsystem 40 may include a number of measurement openings 64, e.g., static pressure and air flow measurement traversal holes, through which the airflow and static pressure can be measured, such as by a static pressure measurement device, e.g., an anemometer and/or a manometer. For instance, a number of the ducting elements 42, 43 can include a one or more static pressure measurement holes, such as positioned in one or more duct elements 42 prior to the air purification module 70, and another one or more static pressure measurement holes, such as positioned in one or more duct elements 43 positioned after the air purification module 70. In this regard, the anemometer may be positioned so that a measurement interface is positioned within the respective holes and the static pressure can thereby be measured.

Likewise, airflow measurement may also be performed in a like manner, except in this instance each duct element wherein a measurement is to be taken may include a plurality of holes, such as five, and a number of measurements, e.g., six, may be performed at each hole. In such an instance, therefore, traversal airflow throughout the system can be determined by taking 30 traversal measurements at each respective measurement duct element, e.g., at two, three, four, or more duct elements. In various embodiments, measurements may be taken periodically, such as prior to aerosolization and/or transmission of the virus into the ducting system 40, e.g., prior to the air purification system 70, and again at a time period post air traversal through the air purification system 70. In this manner static pressure and air flow may be measured across the air purification system 70. However, in various embodiments, the interior cavity of a duct element 42, 43 may include one or more rows of supports, e.g., five, whereby each row includes an internal air flow and/or static pressure measuring device that can communicate wirelessly with the system to measure airflow and static pressure internally.

Nevertheless, regardless of whether the system 1 employs an air handler 15, an air conditioning unit 10, or industrial fan 68, as indicated, the referenced air flow velocities are often too great for particulate matter to be measured accurately, such as without the air flow itself disrupting the measurement process itself. Therefore, an aspect of the innovations herein described, is the design and use of the unique shrouded isokinetic sample probes 60 of FIG. 4. Specifically, in various embodiments, the sample collection probes 60 may include an isokinetic shroud member 63 that is designed to slow the air flowing through the sample reception tube 61 so that when it arrives at the particulate measurement sensor 65, the particulate matter within the air can be measured and quantified, such as with respect to the number and size of particles that have been aerosolized or are otherwise included within the airflow.

Particularly, as can be seen with respect to FIG. 4B, the sample collection probe 60a may be positioned within a cavity 44 of a sample collection duct element 42f of the first ducting module 41a in such a manner that a portion of the air carrying the aerosolized particulate matter may flow across and into the isokinetic shroud 63a. In such instances, the shroud 63a may have a somewhat convoluted air pathway that is designed to slow the air such that as it traverses through the elongated reception tube 61a to the particulate measurement sensor unit 65, it is slowed down enough to be measured therein. Hence, due to the convoluted airflow pathway, the air is slowed, and particulate matter contained therein can be measured, such as with regard to amount, e.g., number, and size of the matter. Then, as can be seen with respect to FIG. 4B, once measured, the air can be returned to the ducting element 42f via a translation conduit 25 so as to continue to be circulated therein and pushed through the air purification module 70 of duct position 42g for purification thereby.

Further, since, in various embodiments, the system is configured for determining the ability of an air purification system 70 to purify the air of airborne biologicals, as well as from volatile organic compounds, and/or other particulate matter, it is often useful to include an additional shrouded isokinetic sample probe 60b, such as post filtration, e.g., so as to once again measure the particulate matter after undergoing the purification and filtration process. Therefore, as can be seen with respect to FIGS. 1 and 2, in particular embodiments, a second unique shrouded isokinetic sample probe 60b can also be provided. Specifically, the second, post-filtration, sample collection probe 60b can be configured in the same or similar manner as the first shrouded isokinetic sample probe 60a. More specifically, the post-filtration sample collection and measurement probe 60b can also include both an isokinetic shroud member 63b for slowing down the air as well as a post filtration particulate measurement sensor 65 for measuring and quantifying the particulate matter within the post-filtration airflow so as to determine the number and size of aerosolized particles that are present within the airflow after that air has been filtered. And similarly, once measured, the air can be returned to the ducting 40 via the translation conduit 25 so as to be circulated therein and pushed through one or more additional post filtration measurement zones.

Accordingly, as can be seen with respect to the methodology set forth in FIG. 5, at Step C, the air containing the aerosolized biological can be directed in through an air purification module 70 so as to be purified thereby. In this regard, as can be seen with respect to FIGS. 14-16, three different air purification modules were tested by the ABATE system presented herein. Particularly, as presented herein below, in order to test the effectiveness and efficiency of a newly developed air purification system 70, such as disclosed in U.S. Pat. Nos. 11,635,221 and 12,111,064, incorporated herein by reference, the present ABATE test systems were developed and used to test three different versions of the air purification systems the basis for which was disclosed therein. For instance, a large, industrial sized air purification unit, a small automotive/aviation air purification unit, and a smaller mobile air purification module, were all used and tested in conjunction with the ABATE system as described herein below.

In all instances, each air purification unit was configured for clarifying the air traversing through the ducting subsystem 40 and into the air purification module 70 of at least 99.99% of VOCs, particles, and viruses. For these purposes, each air purification unit was adaptable to include, one or more, e.g., two, three, four, or more photocatalytic oxidization chambers. In embodiments where multiple chambers were implemented, it was useful for the chambers to be aligned, linearly one right after the other. This configuration was useful because many previous air purification iterations only had a single air purification chamber and appeared not to be adaptable to handling the airflow through the system. However, the present technology has determined that having multiple chambers is useful for fully decomposing harmful VOCs from the air in one or more passes, as explained below.

This configuration was developed specifically for the purpose of deactivating reactive biological agents, such as viruses, and thus, was designed to work in conjunction with a biological, virus, sensor system capable of measuring the quantity and quality of viruses within the air. In this regard, other air purification systems, not designed in this manner, were discouraged from performing actual testing on real-life viruses, e.g., because of bio-safety concerns, and thus, such systems never needed to include a virus sensing system, and were never really designed so as to be capable of deactivating viruses. Accordingly, another innovative aspect of the present disclosure is the development and use of the unique virus sensing systems presented herein.

As indicated, where multiple photocatalytic oxidization chambers were present, each of the chambers were configured so as to be linearly spaced apart from one another. This linear arrangement is useful because it structures the airflow such that a maximal amount of air passes into and through each photocatalytic oxidization chamber in a manner that maximizes deactivation time. Specifically, for these purposes each deactivation chamber may be designed to create an environment wherein volatile organic compounds can undergo a photocatalytic oxidation reaction whereby VOCs may be converted to harmless byproducts such as CO₂ and H₂O. This environment is produced by bombarding a light, such as a UVC LED light, onto a metal oxide panel in the presence of air flow containing the potentially harmful VOCs. Such bombardment generates the requisite photocatalytic oxidization reaction. In particular embodiments, the metal oxide may be embodied by a titanium dioxide mesh panel that is positioned in between two or more UVC generating LED light panels so that the UVC light hits the panel from a multiplicity of sides and a number of different angles. In this manner, the mesh panel may be positioned and/or angled so that the UVC light impinging on that panel assures that all of the flowing air is subjected to this photocatalytic oxidization process.

This configuration is important because as shown in FIGS. 1 and 2, the air purification module 70 is to be inserted within, or in substitution for, a ducting element 42 of the ducting subsystem 40 in line with an air handler 15 or HVAC 10 and should be capable of handling the quantity, directionality, speed, and rate of airflow, while at the same time deactivating both viruses and volatile oxidative compounds, VOCs. Previously implemented air purification systems have not typically been utilized within air ducts as part of the HVAC system, and are not configured in a manner that is capable of handling the velocity of air that flows through the HVAC system. Additionally, as indicated, the present system is unique in that it includes a virus sensor system 60 for determining sensed virus data and measuring the reduction in virus levels and deactivation in the air as it passes through the air purification module 70.

In addition to testing the three referenced indoor air purification modules, as can be seen with respect to FIGS. 1 and 2, a plurality of different configurations of the ABATE ducting system were also tested. Particularly, FIG. 1 presents an ABATE test system in an open-loop, expanded multi-pass configuration, whereas FIG. 9 presents an ABATE test system in an expanded, closed-loop configuration. Likewise, FIGS. 2 and 8 illustrate a double-stacked ABATE test system in a shortened configuration having two sets of rows of ducting elements positioned front and back as well as stacked top and bottom. However, in each different type of configuration of the overall system (single pass vs. multi-pass, stacked vs. double stacked), and for each different type of filtration unit being tested, the ABATE system components may roughly be the same regardless of being in the elongated or shortened and open or closed configurations, because they share the same mechanical characteristics of the airflow.

Further, as illustrated in FIG. 1, the ducting subsystem 40 is in a split orientation so as to emphasize the modular nature of the ducting system. As can be seen, each ducting module 41 is composed of several ducting elements 42 and 43. For instance, the right-hand ducting module 41a includes 8 ducting elements designated 42a, 42b, 42c, 42d, 42e, 42f, 42g, and 42h. Likewise, the left-hand ducting module 41b includes a corresponding 8 ducting elements designated 43a, 43b, 43c, 43d, 43e, 43f, 43g, and 43h. Any number of ducting elements can be included of any sufficient size, depending on the area of the testing environment, and the air flow requirements needed to complete the test.

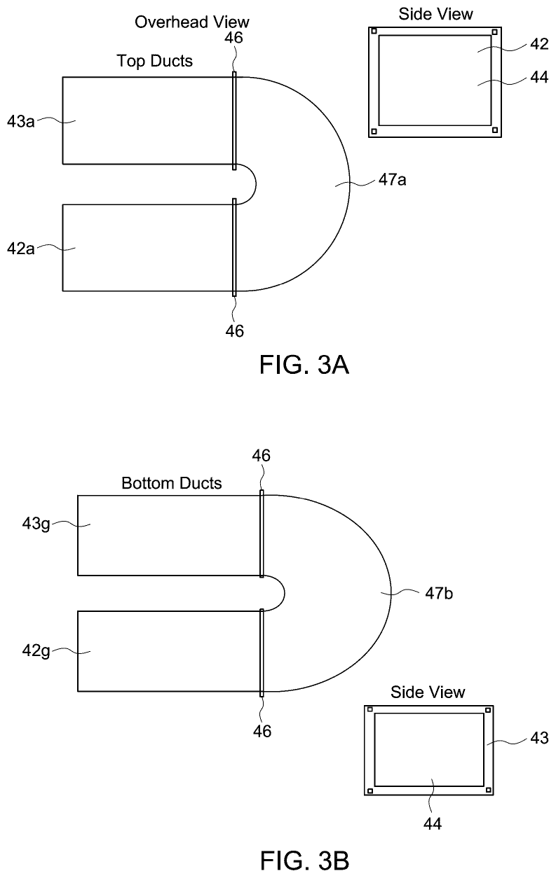

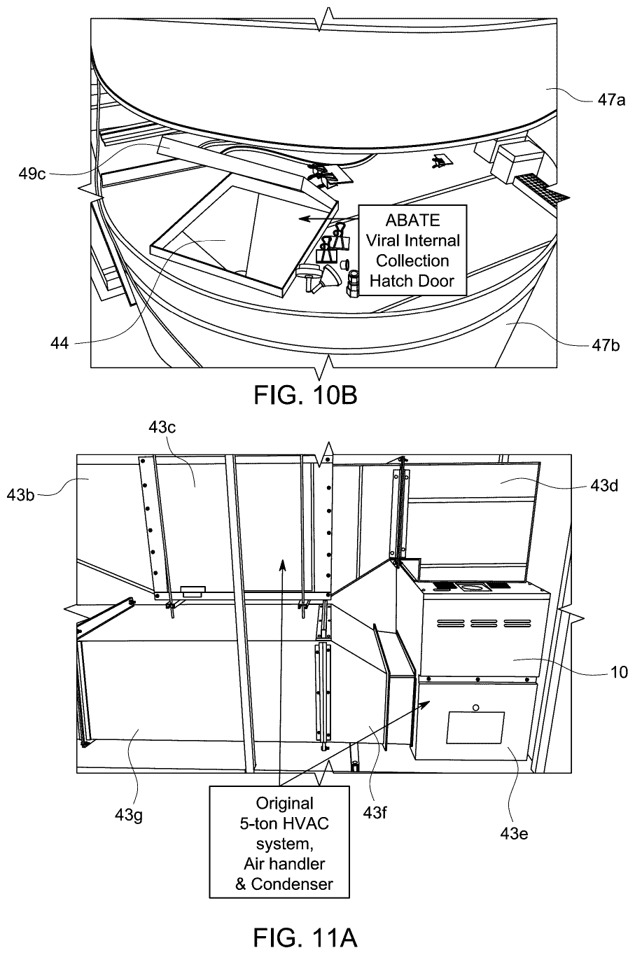

For example, in the depicted embodiments of FIGS. 3A and 3B, the number and sizes of the ducting elements can vary, e.g., dependent on their position within the module 41, but for the rectangular elements 42, they may generally be formed of two pair of opposed walls or panels in the shape of a square or rectangular cube that is open ended so as to allow the air within the ducting to flow through the cavity formed by the four walls. As depicted, each of the walls 45 of the duct elements 42 or 43 may be individually or collectively sized, but in this embodiment, the duct elements may range from about 15 or 18 or 20 or even 25 or about 30 inches in height and/or width, or any increment therebetween, such as about 20¼ or 20⅝ or even 21¾ to about 22½ or 24⅝ or even about 27⅜ in height and/or width, or any combination thereof. It is to be noted that as depicted in FIGS. 2 and 3, a curved duct element 47a and 47b is included. This curve duct configuration is useful because it facilitates a smoother air flow than in those embodiments having a squared 90-degree corner, as in the embodiment of FIG. 1. Thus, airflow velocity is easier to be maintained. Additionally, as can be seen with respect to FIG. 10B, a door 49c can be placed within the ducting element 47b for facilitating sample probe collection, so duct element 47b may also be referenced herein as a collection duct.

Specifically, in particular embodiments tested herein, In the multi-pass configuration described below, utilizing the air handler 15, the typical duct sizes of the ABATE system 1 were about 20″/508 mm high×24″/609.6 mm wide on the bottom row, and 18″/457.2 mm high×20″/508 mm wide on the top row. Likewise, for the single pass configuration, utilizing the commercial fan, the typical duct sizes were 21.5″/546.1 mm high×21.5″/546.1 mm wide on the top row, which quickly turns into a 180° turn into the bottom row of 20″/508 mm high×24″/609.6 mm wide. Further, each of the four walls of the ducts can be coupled together in a manner so as to form an airtight seal with the adjoining duct elements 42, 43, which are configured in like manner. For these purposes, the two duct elements 42 and/or 43 may include a pair of corresponding latching or clasping mechanism for coupling the two or more units together, e.g., linearly.

Any form of coupling mechanism can be employed so as to produce a waterproof and/or air-tight sealing between the two adjoining units. Specifically, the clasping mechanism may be nut and bolt, a weld or rivet, a tongue and groove, corresponding angular teeth like clasping mechanism, or other form of attachment apparatus that effectuates an air-tight seal. In various embodiments, a gasket or seal may be included between the two corresponding attachment interfaces so as two form a waterproof and/or airtight sealing between the two elements. In various embodiments, each attachment interface of the duct elements includes a lip that can be associated with a corresponding lip of the adjoining duct element. In such instances, the two lips can be clipped, welded, riveted, screwed, glued, or otherwise coupled together so as to form an airtight seal. In particular embodiments, the attachment members can be configured to be removably attached so that each ducting module may be easily separated and broken down and easily re-assembled so as to more easily be fit within a laboratory and once therein be assembled. As depicted the corresponding lip elements of adjoining duct elements can be coupled together through a bolt and offset bolt holes. A liquid sealant, such as a silicone sealant, can be sprayed between the two lips to further seal the coupling, or a compressible gasket can be used.

Furthermore, as can be seen with respect to FIG. 1, there are two sets of corner duct elements 42d and 42e, on the left hand-side, and duct elements 43d and 43e on the right-hand side. In this configuration, as air is passed through the ducting elements 42, 43, the air may catch and/or otherwise collect on the corners of the ducting system 40. Consequently, in various embodiments, as can be seen with respect to FIG. 12, in order to ensure maximal flow through the ducting 40, the ABATE system may include one or more turning vanes that are positioned in the corners of the corner duct elements of duct modules 41 a and b. In such embodiments, the turning vanes are configured to ease and direct the flow of air around the corners of the corner duct elements. However, in other embodiments, the duct elements 42d, 42e, 43d, and/or 43e can be rounded and curved so as to approximate the ducts 47a, 47b.

As can be seen with respect to FIG. 1, in various embodiments, one or more of the duct elements 42, 43 (47) can be replaced by, or otherwise contain within the cavity 44 inside it, one or more other system components. It is to be understood that the positioning of the herein referenced other components of the system 1 are depicted as being associated with a location of a particular duct element 42, 43, 47, this positioning may be modified by replacing or otherwise associating the referenced component with a different duct element. This is an advantage of the present modular ducting system 1 in that the various components herein referenced can largely be mixed and matched. Thus, the configuration of the duct elements and any components associated therewith can easily be modified, like LEGGOS® as needed by the space of the contained environment in which the ABATE system is to be deployed, and/or dependent on the testing conditions.

Consequently, in the embodiment set forth in FIG. 1, when looking at ducting module 41a it can be seen that various of the duct elements, in this instance corner duct elements 42d and 42e have been configured so as to include a door 49a and 49b, respectively, positioned therein. These doors are useful for serving as an airflow intake conduit 49b, for taking air into the system, and further for serving as an air exhaust door 49a, for pushing air out of the system, such as in a single pass configuration as depicted in FIG. 9A. Specifically, the opening of these doors is useful when this ducting system 40 is in the single pass configuration, as explained below, while in the multi-pass configuration, these doors should be shut. In various embodiments, the interface between the door 49 and the ducting element 42 includes a compressible gasket that can be compressed when the door 49 is shut so as to thereby create an air and/or waterproof seal thereby. The door may include a latching mechanism, such as a handle and clasp, which secures the door in the closed, single-pass configuration.

Further, the intake door 49b is further useful for manually positioning the nebulization unit 20 within the ducting module 41a such as within duct 42e or 42f. As explained below, the nebulization unit 20 is useful for nebulizing and/or aerosolizing the biological sample, e.g., virus, so as to produce an air borne, e.g., aerosolized, virus that can be transported throughout the ducting subsystem 40 so as to be purified thereby, e.g., by the purification module 70. Consequently, in this instance, because the nebulization unit 20 is positioned within duct element 42f, this duct element may be referred to herein as the nebulization duct. As indicated, within this system set up, it is useful to have the nebulization duct 42f be positioned close to the air purification system 70 so as to facilitate efficient aerosolization of the airborne biological with air, which can then immediately be conveyed through the ducting for air purification thereafter so as to test the effectiveness and efficiency of the air purification unit 70.

In this regard, since the air purification unit is positioned at duct element position 42g, this duct may be referred to herein as the air purification duct 42g. However, as indicated, the positioning of the air purification unit 70 can be variable, such as based on the size of the unit 70 being tested. Consequently, when one of the smaller air purification units is tested, the smaller unit can be positioned at duct element position 42e or 42f. Consequently, wherever the air purification unit 70 is not positioned, a duct element 42 can be positioned in replacement therefore. However, dependent on the configuration of the system 1, the air purification unit 70 can be substituted for virtually any ducting element 42,43, but should be in advance of whichever unit wherein the biological collection duct, in this instance 43f, is positioned. This positioning does not matter as much when the system 1 is in an open loop, multi-pass configuration, as in FIG. 1, but is important in a closed, single pass configuration as in FIG. 9A, which may be in a somewhat linear and/or stacked orientation.

In either a closed or open loop configuration, the air purification duct 42g or 42e may be separated by one, two, three, or more ducting elements, in this instance, duct elements 43h and 43g, from the sample collection duct 43f (47b). Likewise, the air conditioning elements 10, can be substituted for one or more other ducting elements 43, 42, but typically will be positioned post the sample collection duct element 43f. This positioning is useful because it will allow the airflow to be stabilized post the air-conditioning 10 duct position, which in this instance, includes air handling 15 at duct element position 43e, and/or the condenser coil 13, at duct element position 43c.

Particularly, as can be seen with respect to FIG. 1, there are 7 duct elements, e.g., 43b, 43a, 42a, 42b, 42c, 42d, and 42e that separate the air conditioning components from the nebulization components 20. In this manner, the air flow has a sufficient period of length and time to stabilize before coming into contact with the nebulization unit 20. However, it is to be noted that such stabilization is more useful in the open loop, multi-pass configuration, such as where the intake 49b and exhaust 49a doors are closed. In such a multi-pass embodiment, the air can be recycled round and around the ducting subsystem 40, which is useful for safety purposes, such as for preventing the airborne biological from escaping the bounds, e.g., walls, of the ducting elements 42, 43.

Alternatively, in other embodiments, such as that presented with respect to FIGS. 9A, 9B, and 9C, the ABATE system 1 may be configured in a closed loop configuration. For instance, as can be seen with respect to FIG. 9B, the ABATE 1 ducting system 40 is designed to easily be configured for being converted from an open, multi-pass, to a closed, single pass configuration. For instance, the exhaust door 49a can be removed and placed horizontally within the cavities 44 between the exhaust duct element 42d and the air intake duct element 42e. In this manner, the door 49a can be positioned to block the airflow between duct elements 42d and 42e, and thereby close the loop. In this configuration, the intake door 49b can be opened (and removed), and thus, the ducting system 40 can be converted from an open, multi-pass configuration whereby the air flowing through the ducting system 40 can be recycled within the ducting through multiple passes, to a closed loop, single pass configuration, as shown in FIG. 9A.

In such instances, the exhaust duct element 42d can be coupled to the laboratory exhaust for purified air removal, such as after confirmation any airborne biologic has been deactivated, and/or in other instances, one or more transition ducts, as illustrated in FIG. 9C, can be positioned over one or both of the exhaust duct element 42d and/or the air intake duct element 42e, which ducts can then be coupled to one or more of respective laboratory air intake and exhaust. In various embodiments, a filter, e.g., a HEPA and/or a MERV filter can be positioned between the duct and the transition duct and/or between the laboratory exhaust and/or air intake. Such filters can also be used to surround the air purification unit 70. In particular embodiments, the exhaust door 49a may include a sealing member around its boarders, so as to seal the door shut, e.g., in the open-loop configuration, and to seal the passageway shut, e.g., in the closed configuration.

In view of the foregoing, it is to be noted that performing and measuring virus deactivation within a rapid flow of air is not an easy task to accomplish. Part of this difficulty is due to the challenges of performing virus deactivation and air purification within ducting at higher airflow velocities. Another part of this difficulty results from the simple fact that the necessary viral detection sensor systems were not readily available at the time of testing. Hence, prior systems could not fundamentally disclose an actual working sensor system that was capable of detecting a reduction in virus content, because such sensors were not readily available, and had to be specially designed. Thus, outside of the systems and devices disclosed herein, quantitative and qualitative measurement of viral presence and/or deactivation was difficult, if not impossible, to accomplish.

Consequently, for the purposes of performing the real-world testing parameters herein disclosed, the present ABATE system 1 had to be engineered so as to be able to achieve real-world HVAC airflow, while at the same time as being able to deactivate substantially all aerosolized viruses being present within that airflow. Further, in view of the aforementioned sensor difficulties, in order to determine the presence and activity of viruses, VOCs, and/or other particulate matter, flowing at high velocity within the ducting system 40, the unique air purification and sensor configurations disclosed herein had to be specifically designed and developed for these purposes.

Other challenges that had to be overcome in developing a system and methodology for testing the ability of an air purification system 70 to clarify airflow at typical residential and commercial velocities, is the difficulty in measuring the amount of particulate matter being present within the airflow, when that airflow is at higher velocities, such as greater than 1000, greater than 1500, greater than 1800, even greater than 2000 or 3000 or 4000 Cubic Feet per Minute (CFM). As described herein, to overcome these challenges, the present sensor and measurement systems and their methodologies of use were developed. In this regard, the present measurement and sensor systems have been designed to work together to determine the effectiveness and efficiency of the air purification systems disclosed herein to clarify air, namely of harmful viruses, VOCs, and particulate matter, at higher velocities as measured throughout the system. For these purposes, the aforementioned pre- and post-filtration measurement methodologies and sensor probes were developed, and as shown with regard to the testing set forth herein below, they have been shown to overcome these challenges.

Particularly, as can be seen with respect to the configurations set forth with respect to FIGS. 1, 2, and 8, particulate matter, static pressure, airflow, and other, e.g., viral, measurements, described herein, can be taken both at the nebulization point and immediately after the point of filtration. For instance, as can be seen with regard to FIGS. 1 and 2, the aforementioned shrouded isokinetic probes 60a and 60b can be positioned within a pre-filtration duct element, e.g., 42g, 42e, as well as be positioned within a post-filtration duct element 43f, respectively. As discussed above, these probes 60a and 60b are shrouded and positioned within the duct elements so as to allow maximal air flow to pass around the probe, but at the same time as being configured for capturing a sample of that airflow that is then slowed down, e.g., within the shroud, and measured, e.g., by the particulate measurement sensor, with regard to determining the amount and size of particulate matter being present within that airflow.

These measurements are useful because at the beginning of the testing, it is known how much virus is present to be nebulized and/or aerosolized, but it is useful to determine how much of that particulate viral matter actually does get aerosolized, and it is further useful to determine how much of that particulate matter is present so as to be deactivated within the photocatalytic oxidization chamber 75 of the purification system 70. These measurements are useful, therefore, for determining how much virus is present within the airflow as it passes through the air purification module 70. This results in providing a quantification of the ability of the air purification module to deactivate a determined amount of aerosolized virus.

However, as indicated, another important aspect of the processes disclosed herein is ensuring that the aforementioned ability of the air purification module 70 to clarify the airflow at typical operating pressures and air flow as would be experienced within a residence, commercial building, and/or large industrial facility. Consequently, for these purposes, as discussed above with respect to FIG. 8, the ducting system 40 is configured for allowing measurements of static pressure and/or air flow to be taken throughout the system ducting elements 40. For example, such measurements may be taken at the point of nebulization, e.g., within the nebulization duct element 42f, as well as at a post filtration duct element 43. Specifically, in certain embodiments, static pressure can be taken both at the point of nebulization 42f and just prior to filtration 42g, 42e, as well as just after filtration 43h, 43g and/or at the one or more transition duct elements, e.g., 43f.

Likewise, as shown in FIG. 8, airflow measurements may be taken at various points throughout the ducting system 40, such as at a point of exit from one or more transition duct elements, e.g., 43g, but prior to a virus collection duct 47b, as well as approximately midway through the post collection ducting, e.g., 42a, 42b, or 43a, 43b, and the like. As indicated, at each measurement duct, measurements may be taken at number of different positions, e.g., 2, 3, 4, 5 or more, such as where a multiplicity of such measurements, e.g., 2, 3, 4, 5, 6 or more are taken at each position, such as with an anemometer and/or manometer. Hence, in various instances, 30 measurements may be taken, at each measurement duct, over each pass, such as where the various different measurements can be averaged by the anemometer.

Accordingly, regardless of the single-pass or multiple-pass configuration, measurements, e.g., 30 measurements per measurement location, such as for measuring airflow and/or static pressure, can be taken throughout the system at periodic time periods, although in the single pass configuration static pressure should not typically be an issue. However, because airflow naturally varies through different points in the duct it is fair to assume that even with the 30 averaged value used, there would normally be a + or −25 CFM variance per 1000 CFM level. Consequently, in various embodiments, it is useful to perform a plurality of passes so as to take a number of different sets of measurements, over multiple passes, and thereby reduce the effects of such variance.

As introduced above, a key aspect of the innovations set forth herein, is the ability of the ABATE test system 1 to test the ability of the three different disclosed air purification systems 70 to deactivate airborne biologicals as well as to clarify the air of harmful VOCs and other particulate matter. Important features that have been developed and discussed herein above that make such testing possible include the unique design and configurations of the modular airtight ducting subsystem 40, the shrouded particulate matter probes 60, as well as the open and closed airflow configurations of the ducting subsystem 40 that allow for both single- and multi-pass testing and measurement regimes. However, other innovative aspects of the ABATE test system are the novel nebulization subsystem 20, the viral sensing probe system, as well as the quantification and qualification testing methodologies as presented in FIG. 5.

As discussed above, an in relation to the methodology set forth in FIG. 5, a first Step A in the testing process is the growing of the vial load, which may be performed in accordance with well-known viral production methods under safe laboratory conditions, such as at levels 2, 3, 4, or 5, dependent on the nature and virality of the biological being produced. For instance, various viruses can be produced by infecting a cell culture, as shown in FIG. 5, with a virus under growth conditions, letting the virus grow, and then harvesting the virus at the point of cellular lysis. The viruses can then be collected, separated, and be converted into a viral load that is capable of being aerosolized.

Accordingly, at Step B of FIG. 5, the next step in the testing methodology is to aerosolize the biological, e.g., virus, to be flowed through the ducting system 40 and tested. For these purposes, a unique biological, e.g., viral, nebulization system 20 was developed and is set forth and illustrated in FIGS. 6A, 6B, and 6C. Particularly, FIG. 6A presents a biological, e.g., viral, load apparatus for use in accordance with the methods set forth herein. Specifically, the viral load apparatus includes a viral mounting bracket 37 that is configured for retaining the various components of the nebulization system 20 that are adapted for generating a fluidic version of the virus that is capable of being nebulized and/or aerosolized.

In these regards, the mounting bracket 37 is configured for holding the viral loading components in a position for converting the isolated viral load into an aerosolized virus that can be translated through the ducting system 40. For these purposes, the viral mounting bracket 37 is configured for holding a viral load within a viral load tube 32 in a manner that a viral load contained therein can be introduced to and intermixed with a liquid solution, such as of saliva or mucus, or a synthetic version of the same, such as via a viral liquid line manifold 28. For example, upon the viral load being conveyed and contacted with a liquid carrier, e.g., saliva or mucus, such as via translation by a viral holding conduit 33 into a viral loading line 36, the viral load may be contacted and adjoined with the liquid carrier to be intermixed therewith such as by being introduced together in a liquid manifold chamber 28 for intermixing. Particularly, within the liquid manifold 28, the viral-saliva solution can further be intermixed with air, such as via an airline manifold 29, so as to be nebulized and/or aerosolized thereby, as explained below. Consequently, in addition to holding the viral holding tube 32, the viral mounting bracket 37 may also be configured for holding both the viral liquid line manifold 28 in proximity to the airline manifold 29, as well as for holding the various conduit lines interconnecting them.

Specifically, because these nebulization components 20 are adapted for allowing the various different compositions to be intermixed, the viral mounting bracket 37 may further be configured for holding various of the fluid conduits that are used for translating the agents to be nebulized from one chamber to the others to be positioned in close proximity with one another. For instance, the mounting bracket 37 may be adapted for holding the viral load conduit 32 in proximity to both the liquid conduit pressure line 34 and the viral liquid line 36. This orientation is useful because it allows the viral load to be mixed, e.g., under pressure, with air, such as via compressor 30a, and also allows the resultant composition to be further mixed with the carrier liquid via the viral liquid line manifold 28, such as by being translated thereto by the viral liquid line 36. Then as shown with respect to FIG. 6B, the intermixed composition of the virus within the liquid saliva and/or mucus solution may be introduced into the nebulization complex 24 whereby nebulization of the intermixed viral composition may be nebulized and/or aerosolized.

For example, FIG. 6B presents a nebulization complex 24 whereby a viral load may be nebulized and/or aerosolized so as to be capable of being translated throughout the ducting system 40 of the ABATE test system 1, such as for testing the ability of the herein disclosed air purification systems 70 to deactivate the aerosolized live virus and thereby render it harmless. Consequently, for the purposes of generating a nebulized and/or aerosolized viral composition, a unique nebulization complex 24 is herein provided. As can be seen with respect to FIG. 6B, the nebulization complex 24 may include both a viral liquid line manifold 28, as well as an airline manifold 29, together which may be held in proximity to a nebulization unit 26, such as by the viral mounting bracket 27.

In these regards, the viral liquid line manifold 28 and the airline manifold 29 may be coupled to the nebulization unit 26 by one or a plurality of fluid conduits. Particularly, the viral liquid line manifold 28 may be coupled to the nebulization unit 26 via one or more viral liquid lines 36. Likewise, the airline manifold 29 may be coupled to the nebulization unit 26 by one or more airline translation conduits 25. As can be seen with respect to FIG. 6B, there are three independent conduits, e.g., viral liquid lines 36a, 36b, and 36c, for connecting the viral liquid line manifold 28 to the nebulization unit 26. Likewise, there are also three independent air transfer conduits, e.g., air translation lines 25a, 25b, and 25c, for connecting the air line manifold 29 to the nebulization unit 26. This configuration is useful because it allows for a maximal amount of aerosolization to be accomplished in a relatively short time period and within a very confined space.

Hence, another unique feature of the disclosed nebulization system 20, as set forth in FIG. 6C, is a dual port, triple nebulization head unit 21. As can be seen with respect to FIG. 6B, the nebulization complex 24 includes a nebulization head unit 21 that includes three different head ports 21a, 21b, and 21c. Further, as can be seen with respect to FIG. 6C, each nebulization head port 21 may be composed of two reception ports, such as a first, liquid port, for instance, for receiving the liquid viral composition from the viral liquid line manifold 28, via respective viral liquid lines and a second, air-port, such as for receiving pressurized air from the air line manifold 29, via respective air line conduits 25.

Present therein, but not depicted, each nebulization head port 21 may include an individually or collectively controllable solenoid valve, e.g., 27a, 27b, 27c for controlling the translation of the fluidic viral load composition, from the viral liquid manifold 28, and the air from the air line manifold 29, so as to regulate the nebulization process. Consequently, to control the nebulization and/or aerosolization process, as can be seen with respect to FIG. 6D, an electronic and/or digital control unit 21e is provided so as to generate a control command signal that can be communicated to each solenoid valve instructing it when to open and one to close. These operations can be controlled individually or collectively via control features 21f, 21g, and 21h. In particular embodiments, when the solenoid valves 27 are to be controlled collectively, the control features can serve as a spike voltage 21f, a hold voltage 21g, and a control open 21h, as can be seen with reference to FIG. 7A. An on and off switch 21i may also be included. In various embodiments, the solenoid valves can be controlled manually by opening and closing them individually.

Therefore, in use, once activated, respective solenoid valves 27 of the triple head nebulization unit 21 will open, and both the fluidic viral composition will be conveyed to the nebulization head chamber, through the fluid inlet port, via the three respective viral liquid lines 36, and pressurized air will be conveyed to the head chamber, through the gas inlet port, via the three respective air translation conduits 25a, b, c. Once in the nebulization head chamber, the viral fluid and pressurized gas will intermix causing nebulization, and the resultant aerosolized viral composition will be released out through a suitably configured aerosolization exit port and into the ducting of the nebulization duct element, such as via an aspiration process.

Specifically, as can be seen with respect to FIG. 1, the nebulization unit 20 is designed and configured for being removably positioned within one or more of the ducting elements, 42e. For effectuating such purposes, the air intake door 49b may be opened, the nebulization unit 20 may be positioned therein, and the requisite conduits can be attached. Namely, the compressor lines 25a and 25b can be passed through respective openings in the nebulization duct 42f, making sure that an airtight sealing is maintained, such as by an added sealing member, e.g., a gasket or one or more O-rings, being positioned around the openings. Once the compressor lines are connected to their respective couplings with the nebulization unit 20, the cap 38 can be removed from the viral load conduit 32, the virus may then be loaded, the cap 38 can be replaced, and then the intake door 49b can be shut. At this point, in accordance with FIG. 7A, once the nebulization unit 20 has been secured within the nebulization chamber 42f, and the airtight sealings confirmed, then the aerosolization process can be initiated. As can be seen with respect to FIG. 10A, in various embodiments, the door 49b of the nebulization chamber 42e may be or include a transparent member, e.g., a window, so that the nebulization process is viewable from outside of the chamber 44.

For instance, as can be seen with respect to FIG. 7A, the remote-control functionality for the nebulization system 20 is set forth. In this embodiment, the nebulization duct, in this instance, 42f is depicted, with a suitably airtight door being positioned therein providing access to the cavity 44 of the duct. As depicted within the duct 42f, an embodiment of a nebulization system 20 is provided. In this instance, the saliva/mucus composition 23a and the viral load enclosure 23b are positioned outside of the cavity 44 of the nebulization duct 42f, and thus can be loaded, e.g., autoloaded, via a digital and/or electronic controller, while outside of the ducting 44. The viral load enclosure 23b and the saliva/mucus reservoir 23b may be connected by the viral load conduit 32 or viral load conduit 33. In other instances, the two reservoirs may be coupled via the viral liquid line 36.

In this instance, both the viral load and the saliva composition can be controlled via a fluid pump 23, which can take the place of compressor 30a of FIG. 7B. In such an instance, activation of the fluid pump 23 causes one or more of the viral load and the saliva/mucus composition to be released into the viral translation conduits so as to be preliminarily mixed and translated via the viral liquid line 36 into the viral liquid line manifold 28 for mixing. Further, as can be seen, once sufficiently mixed, the viral-saliva composition can be conveyed to the nebulization complex 24 for nebulization thereby.

Specifically, upon turning on the control unit 21e, via power switch 21i, activation of the nebulizer control panel 21 f, g, h will electronically open the solenoid control valves 27, so that the combined viral-saliva solution can be nebulized and/or aerosolized, such as in accordance with Step C of FIG. 5. During aerosolization, the air conditioning unit 10, air handler 15, or fan 68 can be initiated, and the aerosolized virus can be translated through the ABATE ducting subsystem 40 for purification by the air purification system 70. It is to be noted that in accordance with FIG. 7B, the fluid pump 23 can be replaced by a separate compressor 25a for effectuating the mixing and translation of the viral load and/or saliva and/or mucus fluids. In such an instance, the nebulization process may be implemented by a plurality of compressors, a first compressor 30a for running the liquid conduit pressure line 34, such as for translating the viral load, and a second compressor 30b, for running the airlines 25, such as to the airline manifold 29. Also depicted is an anemometer 30d taking measurements.

Further, as can be seen with respect to FIG. 5, once the fluidic vial-saliva composition has been nebulized and/or aerosolized, the aerosolized composition can be conveyed with the air through the air purification system 70 for deactivation thereby. However, as the ABATE system is fundamentally a test environment, once the aerosolized composition has been passed through the air purification module 70, the resulting product can then be sensed, collected, measured, and tested, such as in a designated collection duct 47b of the system 1. For these purposes, as discussed above, a variety of filter containing probes can be placed in the collection duct 43i, 47b, such as for viral collection, which probes can then be removed, and be subject to qualitative and quantitative testing, in accordance with Step D of FIG. 5.

Particularly, as can be seen with respect to FIG. 4C, in one embodiment, a specialized probe 60c has been developed for these purposes. Fundamentally, the probe 60c is similar to the previously discussed particulate matter probes 60a and 60b of FIG. 4B. Specifically, the viral collection probe 60c includes an elongated reception tube 61 having a proximal portion 62a and a distal portion 62b. Similar to the other particle collection probes, the proximal portion 62a may be coupled to a shrouded member 63 that is configured to slow down the air being collected within the reception tube 61. Hence, at both the nebulization 42f and collection points 47b as well as at any other suitable post-filtration point 42/43 g, e, a particulate and/or viral sensor probe 46 can be positioned, such as for detecting and measuring particulate presence and/or virus load, such as before air purification and after air purification.

In this regard, the shrouded portion 63 of the viral probe 60c may include a convoluted and/or tortuous internal pathway for receiving the air therethrough and slowing its delivery to the reception tube 61, whereby the air can then be conveyed to a sample collection assembly 63 that is attached to the distal portion 62b of the viral collection probe 60c. In various embodiments, the entire sample, e.g., viral, collection probe 60c may be positioned entirely within the collection duct 47b. However, in various other embodiments, only the sample collection portion 62a is positioned within the cavity 44 of the collection duct 47b, while the sample measurement portion 62b extends outwards through a wall (or lid) of the collection duct.

In either instance, the distal portion 62b of the sample collection duct 47b may be coupled to a sample collection assembly 63 that is configured for collecting and isolating a portion of the airborne biological, virus, that is retained in the air captured by the sample collection probe 60c. For these purposes, the virus collection assembly 63 may include a collection filter that is removably coupled to the collection assembly 63. In such instances, once a portion of the airborne virus has been collected, the filter containing the collected sample can be removed and subjected to testing, in accordance with Step D of FIG. 5. Additionally, as indicated, a plurality of other probes, e.g., filters, may also be placed in the sample collection duct 47b, and positioned so as to be within the airflow path, such as by being clipped to a cross-sectional support, for additional viral capture. Consequently, as can be seen with respect to FIG. 10B, after aerosolization, purification, and viral capture, the air conditioning unit 10 can be ceased, the collection door 49c within the collection duct 47b can be opened, and all of the probes, e.g., filters, can be collected and subjected to testing, in accordance with Steps D, E, and F of FIG. 5.

Specifically, in accordance with Steps D, E, and F, of FIG. 5, the collected filters can be tested so as to qualitatively and quantitatively examine the particulate matter, e.g., viral matter, collected from the air flow, post-purification and filtration. Particularly, after filtration, at Step D, the viral collection assembly can be accessed, e.g., within the safe confines of the laboratory, and the filter probe can be removed therefrom. The filter can then be tested to determine the quantity of the virus that was collected, for instance, in accordance with known viral quantification methods, such as via RNA isolation and a Reverse Transcriptase-q Polymerase Chain Reaction (qPCR). The results of this assay will give a quantitative measurement that the virus was present, and if so, of how much virus was in the air post filtration. This amount can then be compared to the initial viral load that was aerosolized.

Likewise, in accordance with Steps E and F, once these two filters have been collected, they can be processed, e.g., via a direct CPE assay, so as to determine if the collected virus is alive or dead. Basically, in this assay the viral particles can be collected from the filter and be added into a viable cell culture under conditions such that if live virus were to be present it would infect the cells, and then it could be grown under permissive conditions. After a suitable time period for growth has passed, the cells can be examined to determine if any live sample were present, it would be detected. In this instance, the large presence of live, phenotypical cells would be indicative of any collected virus being present was deactivated, e.g., by the purification process herein described.

Accordingly, as discussed herein throughout, the methodology of FIG. 5 was practiced so as to test three different versions of the referenced air purification system 70. The results of these tests are set forth herein below with reference to FIGS. 13 to 16. As detailed in the graphs provided herein, the line with the lighter (hash) marks represents the content of viruses at the Nebulizer Collection Point and the bold line represents the content of viruses at the Viral Collection Point.

It is to be noted that in the results below that an efficiency level of 99.99% was obtained by each of the referenced indoor air purification systems herein disclosed, which had heretofore not been achievable. Hence, the air purification devices presented and tested herein are unique because the way they are configured allows them to achieve a level of efficiency in killing viruses and removing VOCs and other harmful particulate matter that is vastly improved over previously implemented systems. Accordingly, in all of these iterations, each air purification module included one or more, e.g., three, photocatalytic oxidization chambers, where each of the chambers were configured so as to be linearly spaced apart from one another. Additionally, at the end of the line, the ABATE system 1 included a unique virus sensor system and methodology for employing the same for determining sensed virus data and measuring the reduction in virus levels in the air as it passes through the air purification module 70.

For these purposes, therefore, in a number of the testing described below, a version of an air purification module 70 of the disclosure was provided, such as where the air purification module 70 was configured for generating a photocatalytic oxidization reaction. In these embodiments, the air purification unit was configured to include a number of chambers, such as where each chamber included at least two sets of UVC LED light sources, which were spaced apart from one another to form a treatment zone within each chamber. Further, as can be seen, in particular implementations, one or multiple passes of air through the air purification module 70 was performed to test different device configurations so as to determine their effect on bioaerosols in the airflows.

The testing and results thereof presented herein involved two configurations of the ABATE system 1, as open, e.g., in the single pass configuration, and closed, in the multi-pass configuration. In the single pass iteration, the system was configured as an open system where infectious air flow was drawn through the system once, while in the multi-pass iteration the system formed a closed loop where infectious air flow was recirculated over multiple passes through the system. In particular implementations, such as in the single-pass configuration, the air purification unit included a filter, such as a MERV or HEPA filter. Specifically, as noted in the results below, in particular implementations, a final 24″/609.6 mm×24″/609.6 mm×10″/254 mm HEPA (high efficiency particulate air) filter was positioned at the end of the line, which filter was included both for bio-safety purposes, and to show, as the results set forth below, that even with the HEPA filter present, when the PCO air purification unit was not present, viruses were found to not only be present within the air traversing through the closed system, but they were also found to be active, whereas when the PCO air purification unit was present, both with and without a MERV or HEPA filter, the virus, although still present, was greatly reduced, and all virus were deactivated.

A further complication was determined in that when a HEPA filter was included in the system 1 configuration, a high-powered fan 68 was also required to move air through the filter, which inclusion actually caused the air flow through the air purification unit 70 to decrease. Thus, it was determined that in many instances, the inclusion of a HEPA filter did not deactivate viruses, and required a large industrial sized fan to push air through the system and filter, but at the cost of reducing air flow velocity making this option less than ideal for small and large facilities. Particularly, small facilities cannot readily incorporate a large fan in their HVAC systems, while large facilities need a much higher air flow velocity than is presently capable when including a HEPA filter in the HVAC system. In any of these instances, once through the system 1, the exhausted air was fed into an additional portable air purification unit, for further purification so as to be sure all virus was deactivated prior to collection.

In the multi-pass configuration, on the other hand, the system is in a closed loop configuration where the air and aerosolized Covid-19 is circulated in the closed loop for a set amount of time. In such multi-pass configurations, the test performed were conducted so as to simulate multiple air changes through the system during a multiplicity of timed trials. In the single-pass configuration, the test was set up so that aerosolized virus, e.g., Covid-19, is only sent through the system one time, thereby only allowing the filtration system to be tested on a single pass attempt to deactivate the virus being passed through the system. In this regard, the single-pass test represents the best possible filtration efficacy.

Particularly, if the virus can be deactivated on a single-pass, it will not be infectious after leaving the system. Therefore, the single-pass configuration represents a single ‘choke’ point whereby the virus may be neutralized as the air is circulated through the system. These safety measures were further downstream from the virus collection point and had no bearing on the virus filtration efficacy.

Accordingly, with respect to the virus testing logic and methodology that was performed herein, as set forth above, the testing included aerosolizing viruses, and other associated particulate matter, such as SARS-CoV2 (Covid-19), and various variants thereof, as described above with reference to the nebulization process set forth with respect to FIG. 6. Once aerosolized, the virus, and any associated particulate matter, was propelled through the ABATE system 1 as well as through the different referenced filtration sub-systems.

For instance, in various iterations, one or more, e.g., dual filters, were directly placed within the air stream for each test, and used for additional confirmation. Particularly, in particular iterations, two 47 mm PTFE filters were mounted side by side in line of the air flow, e.g., post flow, so as to concentrate the virus through the 6″/152.4 mm internal transition duct. In some iterations, an additional filter was used for Quantitative Polymerase Chain Reaction (qPCR) testing, with the specially engineered shrouded viral sample collection probe 60c, as described above with reference to FIG. 4.

In certain tests, the two internal filters were used for the performance of Direct Cytopathic Effect (CPE) assays, as described with reference to FIG. 5, Steps D, E, and F. Along with determining the CPE, ozone testing was also performed so as to determine if any harmful byproducts were produced by the PCO reactions taking place in the reactive zones of the chamber. Accordingly, for these purposes a calibrated ozone detector was included as part of the system, and each CPE assay included an ozone air test to ensure that no ozone was produced by the filtration systems. As a result of this testing, it was determined that no ozone was produced by in any of the tests performed.

As can be seen in the results tables, set forth in FIGS. 13 to 16, a series of control tests were performed at the same airflow levels as the different filtration options that were tested. However, the control tests, FIGS. 13A and 13B were performed without any air purification engaged. All testing included performing a series of assays involving a number of viruses, such as Covid-1r9 WT, e.g., the original wild type, as well as the Delta and Omicron variants, which each had a separate Control Test performed. For instance, the various different viruses tested included: 1. Wild-type, clone of SARS-CoV-2, reference strain (2019-nCOV); 2. SA SARS-CoV-2 variant of concern beta (B.1.351; South Africa, May 2020); 3. Delta SARS-CoV-2 variant of concern delta (B.1.617.2; April 2021); 4. Omicron SARS-CoV-2 variant of concern omicron (B.1.1.529; November 2021); 5. BA.2 SARS-CoV-2 variant of concern omicron (B.1.1.529, BA.2; December 2021).

The goal of the efficacy testing was to prove that the disclosed air purification modules, and different tested configurations, could handle the purification of air traversing through an HVAC system serving the largest of commercial buildings. To confirm the results of the testing, a team of 10 independent epidemiologists and engineers reviewed the test procedures and recommended that all testing be carried out with large amounts of viruses to simulate a large number of infected individuals in a large building.

Consequently, the amount of virus that was tested with each and every assay was a 3,000× infectious dose, or enough to infect 3,000 individuals. Further, as described above, to more closely simulate the transmission process of human-to-human infection, prior to aerosolization, the virus and its various variants were mixed with simulated saliva with mucin so as to mimic typical aerosolized breathing and aerosols of human transmission. This mixture included 4 ml of Covid-19/variant and 3 ml of simulated saliva. Volume of virus in milliliters/to the total volume used (brought to total volume with synthetic saliva).

As depicted in FIG. 5, and described herein, for each version of the indoor air purification unit 70 tested, virus was collected mixed with a synthetic saliva, and aerosolized, such as via the nebulizer illustrated in FIGS. 6A and 6B. The fan of the HVAC 10 system was then engaged, such as at about 2,000 CFM, so as to drive the aerosolized virus throughout the ducting of the system 40 and in through the air purification module 70, and finally into the collection filters at collection duct 47b. It is to be noted that in the control version of the testing, e.g., FIGS. 13A and 13b, the indoor air purification module is present, but the UVC light source is not activated, and thus, PCO does not occur. However, for the versions that included purification, once purified and collected, the collection filters were then processed, and a plurality of assays were run on the processed samples, such as for direct CPE and qPCR testing, in accordance with the methodology set forth with respect to FIG. 5.

Particularly, as can be seen with respect to FIGS. 6A-7A, the saliva and virus mixture was aerosolized inside of the ABATE system 1 using the three nozzle heads of FIG. 6C, with pressurized air and liquid lines at 45 psi. As described above, each nebulizing head was controlled with electronics on and shut-off valves, and it typically took between 15 and 45 seconds to fully aerosolize the large dose of virus. The virus aerosolized air was then pushed through by HVAC system 10 within the ducting system 40 and into the air purification, e.g., filtration, unit 70, for virus deactivation and air purification. Likewise, after filtration and decontamination, the air was further pushed through to the collection filters for collection and testing, such as for PCE and qPCR virus detection.

Specifically, each test included 10 different levels of particulate measurements whereby the quantity, e.g., number, mass, and average particulate size were quantified. The aerosols were measured at the point of nebulizing and at the virus final collection points parallel to the point of qPCR collection. Sample data was collected in one second increments during the totality of each test. In this regard, per each test, mass concentration was determined to be: 1. PM 1.0:0.3 to 1.0 μm (microns); 2. PM 2.5:0.3 to 2.5 μm (microns); 3. PM 4.0:0.3 to 4.0 μm (microns); and 4. PM 10:0.3 to 10.0 μm (microns). Likewise, the Number Concentration was determined to be: 1. Part Number 0.5:0.3 to 0.5 μm (microns); 2. Part Number 1.0:0.3 to 1.0 μm (microns); 3. Part Number 2.5:0.3 to 2.5 μm (microns); 4. Part Number 4.0:0.3 to 4.0 μm (microns); and 5. Part Number 10:0.3 to 10.0 μm (microns). This collected data supports the conclusion that the tests were all conducted in a similar manner with similar nebulization.

For the CPE processing, the internal filters were removed and placed directly into separate tubes containing 200 microliter DMEM media, 10% FBS, and penicillin/streptomycin. The media was then plated. Particularly, in performing a cytopathic effect assay, after the liquid media containing the sample is collected, a plate, such as a 96 titer well plate, of living, e.g., permissive, test cells were prepared, elutes from the collection filters are then added to each of the wells and incubated. For instance, the microtiter plate was placed within an incubator, and incubated at 37 C.

After four days of incubation samples were removed and plated, and then placed within a high-resolution microscope for imaging. More particularly, all samples were placed in −80° C. for storage, but 100 microliters were removed from each sample and placed into another tube for back-up. Permissive VeroE6 hACE2 cells were seeded within a microtiter well and 24 hours later frozen collection samples were thawed and the VeroE6 hACE2 cells were subjected to potential infection, i.e., by being mixed with the elutes from the collected filters. Once subjected to potential infection by the collected sample, the Vero E6-hACE2 cells were plated and incubated at 37 C. Detection and imaging were then performed daily using a 4× magnification on a Revolve microscope.

With respect to the results of the cytopathic assay, the cytopathic effect occurs when virus is present and kills permissive cells. Hence, the presence of virus may be noted by the absence of live cells and the clumping of dead cells. For the purposes herein, therefore, CPE was evaluated at day 4 post infection. The presence of the virus was detected by clumping, e.g., representative of dead cells in the plated sample. Such clumping was evidenced only with regard to the control test conditions set forth in reference to FIGS. 13A and 13B. However, as shown with reference to FIGS. 14, 15, and 16, involving PCO filtration, clumping was not observed, and therefore, virus was not present.

Hence, for the results presented in FIGS. 13A and B, the CPE detected sample evidenced large-scale clumping, at d4 post infection, from either or both filter 1 or 2, thereby showing virus was present. As this test was performed with virus aerosolization but without passing through the indoor air purification system, virus presence and infectivity was hypothesized. The results of this control test show although MERV and HEPA filters are effective for removing virus from the air, they cannot and do not remove all viruses from circulating air, and what virus remains, remains active and infectious.

However, on the other hand, as shown in the results with reference to FIGS. 14A, 14B, 15A, 15B, 16A, and 16B, the non-CPE detected sample evidenced no sign of clumping, at d4 post exposure, from either filter 1 or 2. As these tests were performed with virus aerosolization but also with passing the aerosolized virus through the indoor air purification system 70, the deactivation of virus infectivity was hypothesized. For these tests, the ID50 Infectious dose was expressed as ID50, and refers to the number of pathogens sufficient to infect 50% of a given susceptible population. Specifically, CPE not present at d4 post infection indicates <100 PFU SA virus.

Further, as can be seen with respect to FIG. 5, along with the epidemiology CPE testing, described above, a further qPCR assay was conducted so as to authoritatively establish the efficacy of the PCO purifier in deactivating the viruses. In this regard, a singular capture filter was placed in a shrouded capture column next to the particle measurement equipment. Particularly, the capture filter was positioned post nebulization, and hance, after nebulization and transmission, the HVAC was shutdown, the capture filters were removed, the CPE and qPCR assays were run, and the results were processed.

For CPE and qPCR processing the shrouded filter was removed and placed directly into a lysis buffer, and total RNA was extracted. Once extracted, cDNA was synthesized, e.g., using an iscript system. qPCR assay was then performed on the cDNA using CDC validated SARS-CoV2 primer sets in a Taq polymerase system. Specifically, for the performance of Quantitative Polymerase Chain Reaction (qPCR), RNA was first extracted, cDNA was synthesized, and qPCR was run and analyzed.

As indicated, a series of quantitation cycles were then performed in which fluorescence was detected through amplification of the viral cDNA and an analysis thereof was performed. All analysis were performed using the Bio-Rad Maestro software algorithms, e.g., for threshold, and the DNA was plotted and graphed. All qPCR values were based on known DNA standard curve.

Particularly, three different versions of the air purification models disclosed herein were tested. For instance, in a first instance, as depicted in the results of FIGS. 14A and B, in a first iteration, an in-duct air purification module was tested, which module uses multiple layers of protection to filtrate, disinfect, and deactivate. Specifically, in all of the versions tested herein, the air purification modules each included a plurality of air purification elements that were configured for generating a photocatalytic oxidation reaction within an air purification chamber of the device.

In these regards, the air purification elements included all of: two or more UVC light sources, e.g., configured for generating a UVC light, such as germicidal lamps and/or one or more series of UVC LEDs, which light sources were spaced apart from one another so as to form a germicidal treatment zones positioned between the light sources. A PCO-cell (photocatalytic oxidation) panel was positioned within the treatment zone of the chamber and included a plurality of PCO (photocatalytic oxidation) cells, where the PCO cells were configured for interacting with the UVC light in a manner to produce a photocatalytic oxidation reaction within the treatment zone that functions for clarifying air of volatile organic compounds. Additionally, one or more high efficiency particulate filters for trapping the viruses and small particulate matter.

In this version of the air purification modules, the module included a MERV13-4″ thick filter as well as a plurality of photocatalytic oxidation chamber, which chambers each include both a set of UV light sources and a titanium dioxide mesh grid. This configuration is useful because as the UV light shines upon the titanium dioxide mesh, a photocatalytic reaction occurs within the chamber so as to produce agents that function to deactivate viruses and decompose volatile organic compounds. A custom designed MERV13 as well as a custom HEPA filter were also included, where the custom designed MERV13 is configured with ‘Deep Fiber’, and the custom designed Multi-layered HEPA filter was designed to include carbon back and pre-filter. The PCO light source, e.g., UVC Lamp and/or UCC LED, was also specially designed to be employed internally in a manner to decrease the diameter of duct from 20″/508 mm×24″/609.6 mm to 6″/152.4 mm round so as to focus infectious material capture.

In these iterations, the purification system was configured to be installed in HVAC return ducts and is intended to protect buildings by disinfecting the air when the building air is fully exchanged by the air purification module coupled to the HVAC system, commonly measured in ACH (Air changes per hour). As tested, the systems can handle up to about 2,000 or about 2,500 CFM to about 5,000 or 6,000 CFM, such as about 3,000 CFM to about 4,000 CFM, including about 3,500 CFM per unit and can be combined and added together to handle larger airflows and larger HVAC systems. With respect to the present testing, the target for the virus testing is 2,000 CFM +/−75 CFM. Multiple systems, e.g., being used with the 2,000-3,000 CFM target, can be combined to protect a 10,000-20,000 CFM airstream with 5 units combined for example.

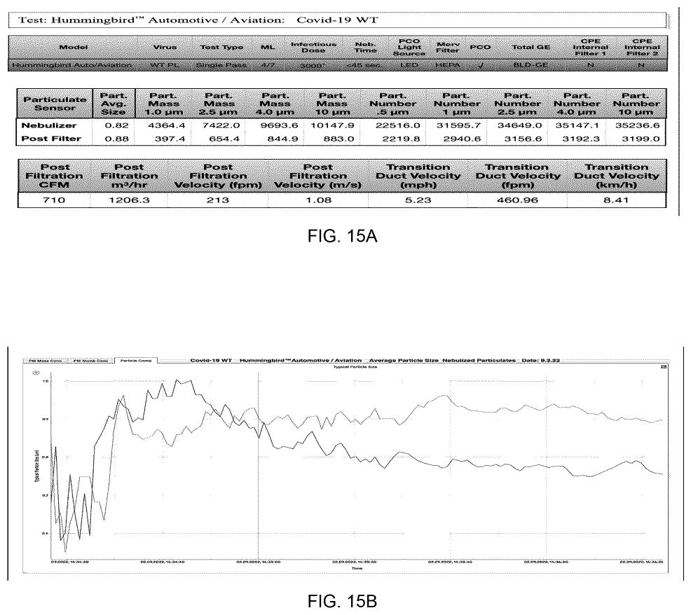

Further, as can be seen with respect to the results of FIGS. 15A and 15B, in a second iteration of the indoor air purification system 70, an automotive and aviation air purification module were tested, which module also uses multiple layers of protection to filtrate, disinfect, and deactivate. In this iteration, the smaller air purification module was configured to be used within an automobile and/or a plane, train, ship, or other conveyance, and is configured so as to disinfecting HVAC airstreams in transportation such as cars, trucks, buses, planes and other vehicles. Specifically, these smaller devices are adapted to be installed in-line with the conveyance HVAC return ducting system and relies on the internal cabin fan to provide the air circulation. It is expected that upon production, the miniaturized modules may directly work with automotive, aviation, sea-faring manufacturers to embed the system in future conveyance production.

Regardless of its smaller size, the module is configured to breaks down VOC's (volatile organic compounds) and carbon monoxide as well as to deactivate viruses. The expected need for transportation systems is to handle up to about 400 to about 600 CFM to about 2,000 CFM, such as from about 1,000 CFM to about 1,500, including about 1,200 CFM of airflows. In this iteration, the system was tested with the ABATE internal fan running at about 700 CFM to meet or exceed the requirements of internal automotive, aviation, and/or sea faring HVAC systems. The active airflow area was 10″/254 mm×10″/254 mm which is similar to the average automotive filter sizes that are approximately 10″/254 mm×9″/228.6 mm. However, the system can be designed to handle any size filter dimension up to about 1,500 to about 2000 CFM, and yet, can also be adapted to service lower CFM such as from about 100 CFM to about 500 CFM, such as about 200 CFM to about 400 CFM, including about 300 CFM.

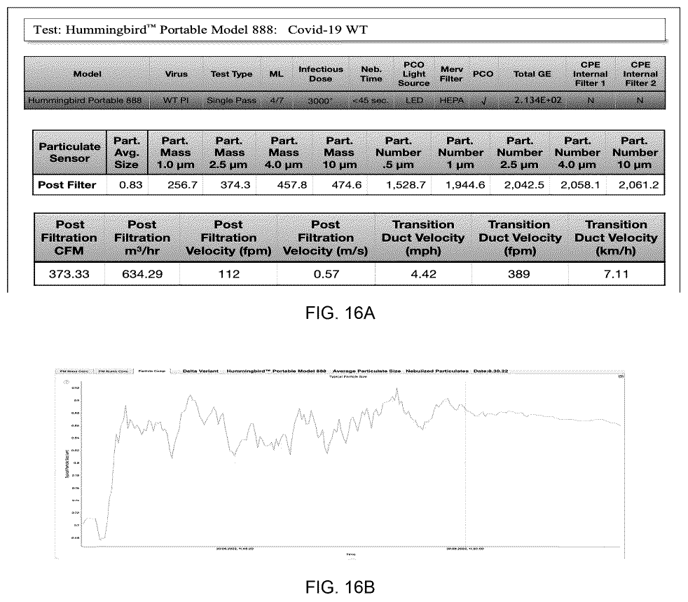

Further still, as can be seen with respect to the results set forth in FIGS. 16A and 16B, a portable module was also tested, such as where the portable module included its own internal fan that was used to circulate the air through the system for decomposition, deactivation, and disinfection. In this version, the system also included multiple filtration levels including a multi-layered MERV and/or HEPA filter, charcoal filters, and/or photocatalytic oxidation. This system was tested inside the ABATE system 1 for biosafety purposes. In this configuration, the internal fan of the module was used for moving airflow, not the ABATE commercial fan.

The results of a portion of these tests are set forth in Tables I-IV, FIGS. 13A, 14A, 15A, and 16A, as well as corresponding Graphs I-IV, see FIGS. 13B, 14B, 15B, and 16B, wherein the Covid-19WT was tested without and with air purification, e.g., filtration, where when filtration was included, such filtration was provided by the three different versions of the indoor air purification modules set forth above.

Accordingly, as can be seen with respect to FIGS. 13A and 13B, a baseline WT virus was aerosolized, transmitted through the system, collected and tested. In the baseline test, 4-7 ml of virus containing solution, with an infectious does of 3000, is aerosolized, e.g., over about 45 seconds, and a single pass is executed, but no air purification was performed. Instead, the aerosolized air is passed through the air purification module, without the PCO reaction being initiated, but the air is, nonetheless, pushed through both MERV and HEPA filters prior to collection.

Hence, the baseline test was conducted with no filtration and was used to compare the baseline results against the different filtration solutions. Specifically, it was noted that for all WT baseline testing, where no PCO initiation was performed, SARS-CoV2/Covid-19 and all variants survived flowing through the HVAC test system at velocities of up to 2,000 CFM in the 20″/508 mm×24″/609.6 mm ducts. Some airflow measurements were above 2,000, CFM but as depicted in Graph I, the average value of 30 measurements were used for all baseline tests which had a similar mechanical setup and profile.

It was noted that the air velocity, which was sped up while traversing the final 6″/152.4 mm to focus the internal transition duct, was over 55 mph/4898 fpm. This velocity is about four times greater than what is currently found in traditional commercial HVAC airflows, and over eight times greater than typical residential HVAC airflows. This strongly indicates that Covid19 and its variants can easily survive while being passed through HVAC systems and are likely spread throughout a building via the HVAC system, regardless of whether a MERV or HEPA filter was present.

Particularly, as depicted in Graph I of FIG. 13B, the bold line indicates the presence of virus present in the air after nebulization, and the lighter dotted line indicates the presence of virus collected in the collection sensor positioned at the end of the pass.

Further, as can be seen with respect to FIGS. 14A and 14B, the same test as in the control above was performed, but in this instance, the aerosolized virus and saliva solution was subjected to photocatalytic oxidization within the chambers of the air purification module. Specifically, the SARS-CoV2 (COVID-19) virus was aerosolized and transmitted into the air purification module such that as the air passes into the PCO chamber, the UV light sources surrounding the chamber focuses UV light upon the titanium dioxide mesh, thereby initiating the PCO reaction within the chamber. The air was then transmitted from the PCO chamber and into the collection filters, which once received within the filters, the HVAC was turned off, the sample was collected from the filters and tested.

Upon assaying, it was determined that the virus was filtered below detection limits in a single-pass test of 5 minutes as compared to the same control test under the same conditions. The results of this testing of the WT SARS-CoV2 were that 99.99% of the virus was deactivated, showing effective decontamination/filtration of the infectious viral particles because of the air being communicated into the air purification module. This effective drop was evidenced even though a vast excess (3000×+) of infectious virus dose was utilized in the aerosolized virus sample. TAs can be seen with respect to Graph II of FIG. 14B, the same results, e.g., 99.99% or greater effective decontamination/filtration of infectious viral particles, was evidenced when testing all of: Delta SARS-CoV2, Omicron SARS-CoV2, and Omicron BA.2 SARS-CoV2. These results show that the filtration system inactivates all of these variants.

Furthermore, as can be seen with respect to FIGS. 15A and 15B, a further air purification device was also tested for its ability to deactivate/filter SARS-CoV2 (COVID-19) to below detection limits, in a single-pass test, e.g., of 5 minutes, as compared to the control test under the same conditions. In this regard, the actual air purification device that was tested here was configured to be much smaller than the device tested above, such that it was dimensioned so as to fit within a car, plane, train, sea going vessel, or other smaller dimensioned conveyance, including even an elevator and the like. In correspondence with the test results of the air purification device above, the smaller automotive/aviation device was found to be 99.99% effective for decontaminating the air of infectious viral particles as well as deactivating the virus. In this regard, the 99.99% effective drop was evidenced even when using a vast excess (3000×+) of virus necessary for an infectious dose, and was effective against all of WT SARS-CoV2, Delta SARS-CoV2, Omicron SARS-CoV2, and Omicron BA.2 SARS-CoV2 viruses.

Additionally, as can be seen with respect to the results presented in FIGS. 16A and 16B, an additional air purification device was also tested, such as where the device was configured to be even smaller than the automotive/aviation device above, and was, in fact, dimensioned so as to be easily portable. The results of this testing showed that the portable air purification device was effective for deactivating/filtering WT SARSCoV2 (COVID-19) to below detection limits in a single-pass test of 5 minutes as compared to the same control test under the same conditions.

Specifically, corresponding to the above, the small portable device was found to be 99.99% effective for decontaminating the air of infectious viral particles as well as deactivating the virus. And, like above, the device evidenced a 99.99% effective drop in viral load even when using a vast excess (3000×+) of virus necessary for an infectious dose. The portable device was further found to be 99.99% effective against all of WT SARS-CoV2, Delta SARS-CoV2, Omicron SARS-CoV2, and Omicron BA.2 SARS-CoV2, suggesting that the filtration system inactivates the WT as well as all of the variants.

Accordingly, as can be seen from the above, three differently dimensioned air purification devices were tested to determine their ability to clarify the air of viruses as well as to deactivate viruses, such as, the original COVID as well as the Delta, Omicron, and BA2 variants. The tables and graphs above show the results of all the tests as well as the respective conditions, including the airflow, the number of particulates that were put into the air, and their mass. In all but the control testing, the air was substantially clarified of virus after one pass through the purification unit, and all viruses were deactivated.

Accordingly, in view of the above, in one aspect, the present disclosure is directed to a system for testing one or more of an intelligent HVAC and/or Air Purification system, which in various embodiments, may be configured for removing and testing the removal of harmful airborne biologicals, such as viruses, from the air traversing through an air conditioning duct system. In various embodiments, the system may include one or more of a structured database, one or more client computing devices, and one or more server systems, such as to track and/or control the operations and results of the testing. For instance, the results of the testing as well as the testing parameters may be stored and tracked within a structured database, such as a structured database that includes one or more searchable libraries, for example, a database wherein at least one library contains data files pertaining to one or more, e.g., a plurality, of optimal operational parameters of the devices of the system for producing the desired environmental conditions, and another library that includes a description for one or more, e.g., a plurality of environmental and/or user health conditions.

Further, in various embodiments, the system may include a client computing device having a display coupled therewith, such as for displaying a graphical user interface that is generated by one or more of the client computing device and/or a server associated therewith, such as where the computing system determines the results of the testing and performs one or more analysis thereon. For example, in a particular embodiment, a testing environment in need of monitoring, conditioning, and/or maintenance may be defined, its conditions determined, and an ideal testing environmental condition for one or more tests being run therein may be determined. Accordingly, a user, such as a system administrator, may engage the user interface of the client computing device for the purpose of defining one or more tests objectives and/or conditions that may be useful for achieving one or more goals of one or more test administrators.

Particularly, an individual, e.g., system administrator, may access one or more controls of an interactive dashboard presented at the graphical user interface so as to participate in an test condition interview, whereby the individual's response to one or more intuitive interview questions may be collected by the system and analyzed thereby so as to determine an optimal test condition for an environment, such as with respect to testing the performance of an air purification module. For instance, the GUI presented at the display of the client computing device may be configured for displaying a list of interview questions and/or test condition characteristics to the user, for receiving, such as via one or more controls of a dashboard interface, the individual's responses, selections, and/or instructions to the interview questions, which once received the responses may then be transmitted, such as by a suitably configured communications module to an associated server system.

The system, therefore, may include a server system, such as a server that is connected to one or both of the database and the client computing device via a network connection. Particularly, in various implementations, the server system may be configured for generating an test builder module, which test builder may be adapted for providing the referenced graphical user interface to the client computing device for local display thereby. More particularly, in certain instances, the client computing device may be used by a system administrator for generating and/or selecting operational parameters for configuring the various system test apparatuses of the system. However, in particular instances, the server system may be employed, or otherwise configured to autonomously generate the conditions, such as in an intuitive manner.

Embodiments of the disclosure and all of the functional operations described in this specification can be implemented in digital electronic circuitry, or in computer software, firmware, or hardware, including the structures disclosed in this specification and their structural equivalents, or in combinations of them. Embodiments of the disclosure can be implemented as one or more computer program products, e.g., one or more modules of computer program instructions encoded on a computer readable medium, e.g., a machine readable storage device, a machine readable storage medium, a memory device, or a machine-readable propagated signal, for execution by, or to control the operation of, data processing apparatus.

The term “data processor” or “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of them. A propagated signal is an artificially generated signal, e.g., a machine-generated electrical, optical, or electromagnetic signal, that is generated to encode information for transmission to suitable receiver apparatus.

A computer program (also referred to as a program, software, an application, a software application, a mobile application, a script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit). Processors suitable for the execution of a computer program include, by way of example, both general and special purpose microprocessors, and any one or more processors of any kind of digital computer.

Generally, a processor will receive instructions and data from a read only memory or a random-access memory or both. The essential elements of a computer are a processor for executing instructions and one or more memory devices for storing instructions and data. Typically, a computer will also include, or be operatively coupled to, a communication interface to receive data from or transfer data to, or both, one or more mass storage devices for storing data, e.g., magnetic, magneto optical disks, or optical disks. Moreover, a computer can be embedded in another device, e.g., a mobile telephone, a personal digital assistant (PDA), a mobile audio player, a Global Positioning System (GPS) receiver, to name just a few.

Information carriers suitable for embodying computer program instructions and data include all forms of non-volatile memory, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, embodiments of the disclosure can be implemented on a computer having a display device, e.g., a capacitive sensing touch screen device, including a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

Embodiments of the disclosure can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the invention, or any combination of such back end, middleware, or front-end components. The components of the system can be interconnected by any form or medium of digital data communication, e.g., a communication network. Examples of communication networks include a local area network (“LAN”) and a wide area network (“WAN”), e.g., the Internet. The computing system can include clients and servers. A client and server are generally remote from each other and typically interact through a communication network. The relationship of client and server arises by virtue of computer programs running on the respective computers and having a client-server relationship to each other.

Certain features which, for clarity, are described in this specification in the context of separate embodiments, may also be provided in combination in a single embodiment. Conversely, various features which, for brevity, are described in the context of a single embodiment, may also be provided in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Particular embodiments of the invention have been described. Other embodiments are within the scope of the following claims. For example, the steps recited in the claims can be performed in a different order and still achieve desirable results. In addition, embodiments of the invention are not limited to database architectures that are relational; for example, the invention can be implemented to provide indexing and archiving methods and systems for databases built on models other than the relational model, e.g., navigational databases or object-oriented databases, and for databases having records with complex attribute structures, e.g., object oriented programming objects or markup language documents. The processes described may be implemented by applications specifically performing archiving and retrieval functions or embedded within other applications.

Although a few embodiments have been described in detail above, other modifications are possible. Other embodiments may be within the scope of the following claims.

1. An airborne biological assessment threat elimination system for collecting and measuring airborne biologicals, the system comprising:
an intelligent heating, ventilation, and air conditioning (HVAC) unit for efficiently purifying air of a compartmentalized environment in a manner that deactivates airborne biologicals, the HVAC unit comprising:
a nebulization unit comprising a plurality of chambers, a fluid pump, as well as a plurality of translation conduits, the plurality of chambers including a biological load chamber and a nebulization chamber, the biological load chamber being coupled to the nebulization chamber by a first translation conduit, and being configured for retaining a liquid load of the biological, the fluid pump for delivering the liquid biological load to the nebulization chamber, wherein the nebulization chamber nebulizes the liquid biological load to produce a nebulized biological load;
a compressor unit being coupled to the nebulization unit via a second translation conduit, and being configured for both converting the nebulized biological load into an aerosolized biological load and for delivery via a third translation conduit;
a modular ducting subsystem being coupled to the nebulization unit by the third translation conduit, the modular ducting subsystem comprising a first and a second ducting module, each ducting module including at least two segments of one or more duct elements, at least one of the one or more duct elements of each ducting module comprising a sample collection duct element, each duct element being composed of a pair of opposed walls defining a cavity therebetween, the two segments having respective coupling mechanisms that are adapted for removably joining respective duct elements of the two segments together in airtight sealings, the sample collection duct element of the first ducting module being configured for receiving the aerosolized biological load from the nebulization unit via the third translation conduit, and further being configured for directing a first portion of the aerosolized biological load through the first ducting module and into the sample collection duct element of the second ducting module in a manner whereby neither air nor one or more portions of the aerosolized biological load can escape through the airtight sealings, the first sample collection duct element of the second ducting module being configured for receiving a second portion of the aerosolized biological load;
a first sampling probe coupled to the sample collection duct element of the first ducting module, the first sampling probe having a first elongated reception tube, the first reception tube including a first portion and a second portion, the first portion extending inwards from a first of the opposed walls of the sample collection duct element of the first ducting module, into a cavity defined thereby, and being coupled to a first capture assembly for capturing a first amount of the first portion of the aerosolized biological load so as to produce a first biological load sample, and the second portion of the first reception tube being positioned so as to extend at least partially outside of the first wall, the second portion of the first reception tube being adapted for allowing collection of the first biological load sample;
an air purification module positioned between the first ducting module and the second ducting module, the air purification module being configured for receiving an uncaptured portion of the aerosolized biological load, for purifying the uncaptured portion of the aerosolized biological load so as to produce the second portion of the aerosolized biological load, and for translating the second portion of the aerosolized biological load into the sample collection duct element of the second ducting module; and
a second sampling probe coupled to the sample collection duct element of the second ducting module, the second sampling probe having a second elongated reception tube, the second reception tube including a first portion and a second portion, the first portion extending inwards from a first of the opposed walls of the second sample collection duct element into a cavity defined thereby and being coupled to a second capture assembly for capturing a first amount of the second portion of the aerosolized biological load so as to produce a second biological load sample, and the second portion of the second reception tube being positioned so as to extend at least partially outside of the first wall, the second portion for allowing collection of the second biological load sample.

2. The system in accordance with claim 1, wherein the first and second sampling probes are both configured for slowing a flow of air containing the portion of the aerosolized biological load to be captured.

3. The system in accordance with claim 2, wherein one or more of the first and second sampling probe comprises a shrouded isokinetic tube.

4. The system in accordance with claim 3, wherein the first and second biological load samples comprise a viral load, and the second biological load sample, having passed through the air purification module, comprises a viral load that is 99.99% deactivated as compared to the first biological load sample.

5. The system in accordance with claim 4, wherein the compartmentalized environment comprises a level 2, 3, or 4 biosafety laboratory.

6. The system in accordance with claim 5, further comprising a fan configured for being positioned in line with one or more of the first and a second ducting modules, the fan being configured for generating an airflow sufficient to propel the first and second biological load throughout the modular ducting subsystem.

7. The system in accordance with claim 6, wherein the air flow comprises a flow between 2,000 CFM to 3,000 CFM.

8. The system in accordance with claim 7, wherein the nebulizer further comprises a single nebulizer head having a plurality of ports, each port being configured to receive a portion of the liquid load of the biological as well as for receiving translated air under pressure from the compressor for the purpose of aerosolizing the nebulized biological.

9. The system in accordance with claim 8, wherein each nebulizer port includes an individual solenoid valve for collectively or independently controlling pressure and flow through respective nebulizer ports.

10. The system in accordance with claim 9, wherein the compressor unit comprises a plurality of compressors, a first compressor for producing air flow in one or more gas translation conduits, and a second compressor for producing air flow in one or more liquid translation conduits.

11. The system in accordance with claim 10, wherein the HVAC unit further comprises one or more of an air-handler, air conditioner, furnace, and condenser coil.

12. The system in accordance with claim 11, wherein each ducting module includes a plurality of rows of stacked duct elements forming a loop in the shape of a “U”.

13. The system in accordance with claim 12, wherein each ducting module includes up to about 25 feet of duct elements.

14. The system in accordance with claim 12, wherein each ducting module includes a plurality of double stacked rows of duct elements, the double stacked rows including two top rows and two bottom rows, one row in front and one row in back, whereby the two top and bottom rows are coupled together by a pair of respective curved duct elements.

15. The system in accordance with claim 14, wherein each curved duct element comprises a curvature of about 180 degrees, and is configured to couple each front row with each respective back row.
